SEQUENCE LISTING (69 sequences)

SEQ ID NO: 1 - protein, 354 aa, synthetic construct
  REGION 1..354: note = HC of antibody PR001046, Chothia definition
EVQLVETGGGLIQPGGSLRLSCAASGFTVSDNYMTWVRQAPGKGLEWVSVIFSGGNTYYADSVKGRFTISRDNAKNTLYLQMNSLRAEDTALYYCARRNYDDTRGTDVFDIWGQGTMVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 451 aa, synthetic construct
  REGION 1..451: note = HC of antibody PR000274 , Chothia definition
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 453 aa, synthetic construct
  REGION 1..453: note = HC1 of antibody PR002199, Chothia definition
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 455 aa, synthetic construct
  REGION 1..455: note = HC1 of antibody PR002299, Chothia definition
EVQLLESGGGLVQPGGSLRLSCAASGFTFSTYAMNWVRQAPGKGLEWVSRIRSKYNNYATYYADSVKDRFTISRDDSKSTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 455 aa, synthetic construct
  REGION 1..455: note = HC1 of antibody PR003178, Chothia definition
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 477 aa, synthetic construct
  REGION 1..477: note = HC2 of antibody PR002199, Chothia definition
EVQLLESGGGLVQPGGSLRLSCAASGFTVSSYGMSWVRQAPGKGPEWVSGIRGSDGSTYYADSVKGRFTISRDNSKNTLYLQMNSLRAEDTAVYYCAKQGENDGPFDHRGQGTLVTVSSGGGGSEVQLLESGGGLVQPGGSLRLSCAASGFTVSSYGMSWVRQAPGKGPEWVSGIRGSDGSTYYADSVKGRFTISRDNSKNTLYLQMNSLRAEDTAVYYCAKQGENDGPFDHRGQGTLVTVSSASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 7 - protein, 487 aa, synthetic construct
  REGION 1..487: note = HC2 of antibody PR002299, Chothia definition
EVQLLESGGGLVQPGGSLRLSCAASGFTFSSYAMIWVRQAPGKGLEWVSGISESGGSTYYADSVKGRFTISRDNSKNTLYLQMNSLRAEDTAVYFCVKDLDDILTGYYKDYWGQGTLVTVSSRTGGGGSKLEVQLLESGGGLVQPGGSLRLSCAASGFTFSSYAMIWVRQAPGKGLEWVSGISESGGSTYYADSVKGRFTISRDNSKNTLYLQMNSLRAEDTAVYFCVKDLDDILTGYYKDYWGQGTLVTVSSASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 8 - protein, 493 aa, synthetic construct
  REGION 1..493: note = HC2 of antibody PR003178, Chothia definition
EVQLVETGGGLIQPGGSLRLSCAASGFTVSDNYMTWVRQAPGKGLEWVSVIFSGGNTYYADSVKGRFTISRDNAKNTLYLQMNSLRAEDTALYYCARRNYDDTRGTDVFDIWGQGTMVTVSSGGGGSGGGGSGGGGSEVQLVETGGGLIQPGGSLRLSCAASGFTVSDNYMTWVRQAPGKGLEWVSVIFSGGNTYYADSVKGRFTISRDNAKNTLYLQMNSLRAEDTALYYCARRNYDDTRGTDVFDIWGQGTMVTVSSASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 9 - protein, 5 aa, synthetic construct
  REGION 1..5: note = HCDR1 of antibody PR000274, Kabat definition
NYWMH

SEQ ID NO: 10 - protein, 5 aa, synthetic construct
  REGION 1..5: note = HCDR1 of antibody PR001046 or VH2 HCDR1 of antibody PR003178, Kabat definition
DNYMT

SEQ ID NO: 11 - protein, 5 aa, synthetic construct
  REGION 1..5: note = VH1 HCDR1 of antibody PR002199, Kabat definition
DYAMH

SEQ ID NO: 12 - protein, 5 aa, synthetic construct
  REGION 1..5: note = VH2 HCDR1 of antibody PR002199, Kabat definition
SYGMS

SEQ ID NO: 13 - protein, 5 aa, synthetic construct
  REGION 1..5: note = VH1 HCDR1 of antibody PR002299 or PR003178, Kabat definition
TYAMN

SEQ ID NO: 14 - protein, 5 aa, synthetic construct
  REGION 1..5: note = VH2 HCDR1 of antibody PR002299, Kabat definition
SYAMI

SEQ ID NO: 15 - protein, 7 aa, synthetic construct
  REGION 1..7: note = HCDR1 of antibody PR000274, Chothia definition
GGTFSNY

SEQ ID NO: 16 - protein, 7 aa, synthetic construct
  REGION 1..7: note = HCDR1 of antibody PR001046 or VH2 HCDR1 of PR003178, Chothia definition
GFTVSDN

SEQ ID NO: 17 - protein, 7 aa, synthetic construct
  REGION 1..7: note = VH1 HCDR1 of antibody PR002199, Chothia definition
GFTFDDY

SEQ ID NO: 18 - protein, 7 aa, synthetic construct
  REGION 1..7: note = VH2 HCDR1 of antibody PR002199, Chothia definition
GFTVSSY

SEQ ID NO: 19 - protein, 7 aa, synthetic construct
  REGION 1..7: note = VH1 HCDR1 of antibody PR002299 or antibody PR003178, Chothia definition
GFTFSTY

SEQ ID NO: 20 - protein, 7 aa, synthetic construct
  REGION 1..7: note = VH2 HCDR1 of antibody PR002299, Chothia definition
GFTFSSY

SEQ ID NO: 21 - protein, 10 aa, synthetic construct
  REGION 1..10: note = HCDR1 of antibody PR000274, Combined definition
GGTFSNYWMH

SEQ ID NO: 22 - protein, 10 aa, synthetic construct
  REGION 1..10: note = HCDR1 of antibody PR001046 or VH2 HCDR1 of antibody PR003178, Combined definition
GFTVSDNYMT

SEQ ID NO: 23 - protein, 10 aa, synthetic construct
  REGION 1..10: note = VH1 HCDR1 of antibody PR002199, Combined definition
GFTFDDYAMH

SEQ ID NO: 24 - protein, 10 aa, synthetic construct
  REGION 1..10: note = VH2 HCDR1 of antibody PR002199, Combined definition
GFTVSSYGMS

SEQ ID NO: 25 - protein, 10 aa, synthetic construct
  REGION 1..10: note = VH1 HCDR1 of antibody PR002299 or PR003178, Combined definition
GFTFSTYAMN

SEQ ID NO: 26 - protein, 10 aa, synthetic construct
  REGION 1..10: note = VH2 HCDR1 of antibody PR002299, Combined definition
GFTFSSYAMI

SEQ ID NO: 27 - protein, 5 aa, synthetic construct
  REGION 1..5: note = HCDR2 of antibody PR001046 or VH2 HCDR2 of PR003178, Chothia definition
FSGGN

SEQ ID NO: 28 - protein, 6 aa, synthetic construct
  REGION 1..6: note = HCDR2 of antibody PR000274, Chothia definition
YRGHSD

SEQ ID NO: 29 - protein, 6 aa, synthetic construct
  REGION 1..6: note = VH1 HCDR2 of antibody PR002199, Chothia definition
SWNSGS

SEQ ID NO: 30 - protein, 6 aa, synthetic construct
  REGION 1..6: note = VH2 HCDR2 of antibody PR002199, Chothia definition
RGSDGS

SEQ ID NO: 31 - protein, 6 aa, synthetic construct
  REGION 1..6: note = VH2 HCDR2 of antibody PR002299, Chothia definition
SESGGS

SEQ ID NO: 32 - protein, 8 aa, synthetic construct
  REGION 1..8: note = VH1 HCDR2 of antibody PR002299 or PR003178, Chothia definition
RSKYNNYA

SEQ ID NO: 33 - protein, 16 aa, synthetic construct
  REGION 1..16: note = HCDR2 of antibody PR001046 or VH2 HCDR2 of antibody PR003178, Combined or Kabat definition
VIFSGGNTYYADSVKG

SEQ ID NO: 34 - protein, 17 aa, synthetic construct
  REGION 1..17: note = HCDR2 of antibody PR000274, Combined or Kabat definition
ATYRGHSDTYYNQKFKG

SEQ ID NO: 35 - protein, 17 aa, synthetic construct
  REGION 1..17: note = VH1 HCDR2 of antibody PR002199, Combined or Kabat definition
GISWNSGSIGYADSVKG

SEQ ID NO: 36 - protein, 17 aa, synthetic construct
  REGION 1..17: note = VH2 HCDR2 of antibody PR002199, Combined or Kabat definition
GIRGSDGSTYYADSVKG

SEQ ID NO: 37 - protein, 17 aa, synthetic construct
  REGION 1..17: note = VH2 HCDR2 of antibody PR002299, Combined or Kabat definition
GISESGGSTYYADSVKG

SEQ ID NO: 38 - protein, 19 aa, synthetic construct
  REGION 1..19: note = VH1 HCDR2 of antibody PR002299 or PR003178, Combined or Kabat definition
RIRSKYNNYATYYADSVKD

SEQ ID NO: 39 - protein, 10 aa, synthetic construct
  REGION 1..10: note = VH2 HCDR3 of antibody PR002199, Chothia, Combined or Kabat definition
QGENDGPFDH

SEQ ID NO: 40 - protein, 12 aa, synthetic construct
  REGION 1..12: note = HCDR3 of antibody PR000274, Chothia, Combined or Kabat definition
GAIYDGYDVLDN

SEQ ID NO: 41 - protein, 13 aa, synthetic construct
  REGION 1..13: note = VH2 HCDR3 of antibody PR002299, Chothia, Combined or Kabat definition
DLDDILTGYYKDY

SEQ ID NO: 42 - protein, 14 aa, synthetic construct
  REGION 1..14: note = HCDR3 of antibody PR001046 or VH2 HCDR3 of antibody PR003178 , Chothia, Combined or Kabat definition
RNYDDTRGTDVFDI

SEQ ID NO: 43 - protein, 14 aa, synthetic construct
  REGION 1..14: note = VH1 HCDR3 of antibody PR002199, Chothia, Combined or Kabat definition
DSRGYGDYRLGGAY

SEQ ID NO: 44 - protein, 14 aa, synthetic construct
  REGION 1..14: note = VH1 HCDR3 of antibody PR002299 or antibody PR003178, Chothia, Combined or Kabat definition
HGNFGNSYVSWFAY

SEQ ID NO: 45 - protein, 214 aa, synthetic construct
  REGION 1..214: note = LC of antibody PR000274, Chothia definition
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 214 aa, synthetic construct
  REGION 1..214: note = LC of antibody PR002199, Chothia definition
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 215 aa, synthetic construct
  REGION 1..215: note = LC of antibody PR002299 or PR003178, Chothia definition
QAVVTQEPSLTVSPGGTVTLTCRSSTGAVTTSNYANWVQQKPGQAPRGLIGGTNKRAPWTPARFSGSLLGDKAALTLLGAQPEDEAEYFCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 48 - protein, 11 aa, synthetic construct
  REGION 1..11: note = VL LCDR1 of antibody PR000274, Chothia, Combined or Kabat definition
SASQDISNYLN

SEQ ID NO: 49 - protein, 11 aa, synthetic construct
  REGION 1..11: note = VL LCDR1 of antibody PR002199, Chothia, Combined or Kabat definition
RASQSVSSNLA

SEQ ID NO: 50 - protein, 14 aa, synthetic construct
  REGION 1..14: note = VL LCDR1 of antibody PR002299 or antibody PR003178, Chothia, Combined or Kabat definition
RSSTGAVTTSNYAN

SEQ ID NO: 51 - protein, 7 aa, synthetic construct
  REGION 1..7: note = VL LCDR2 of antibody PR000274, Chothia, Combined or Kabat definition
YTSNLHS

SEQ ID NO: 52 - protein, 7 aa, synthetic construct
  REGION 1..7: note = VL LCDR2 of antibody PR002199, Chothia, Combined or Kabat definition
GASTRAT

SEQ ID NO: 53 - protein, 7 aa, synthetic construct
  REGION 1..7: note = VL LCDR2 of antibody PR002299, or VL LCDR2 of PR003178, Chothia, Combined or Kabat definition
GTNKRAP

SEQ ID NO: 54 - protein, 9 aa, synthetic construct
  REGION 1..9: note = VL LCDR3 of antibody PR000274, Chothia definitionVL LCDR3 of PR000274, Combined definitionVL LCDR3 of PR000274, Kabat definition
QQYRKLPWT

SEQ ID NO: 55 - protein, 9 aa, synthetic construct
  REGION 1..9: note = VL LCDR3 of antibody PR002199, Chothia, Combined or Kabat definition
QQYNNWPWT

SEQ ID NO: 56 - protein, 9 aa, synthetic construct
  REGION 1..9: note = VL LCDR3 of antibody PR002299 or antibody PR003178, Chothia, Combined or Kabat definition
ALWYSNLWV

SEQ ID NO: 57 - protein, 119 aa, synthetic construct
  REGION 1..119: note = VH2 of antibody PR002199, Chothia definition
EVQLLESGGGLVQPGGSLRLSCAASGFTVSSYGMSWVRQAPGKGPEWVSGIRGSDGSTYYADSVKGRFTISRDNSKNTLYLQMNSLRAEDTAVYYCAKQGENDGPFDHRGQGTLVTVSS

SEQ ID NO: 58 - protein, 121 aa, synthetic construct
  REGION 1..121: note = VH of antibody PR000274, Chothia definition
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSS

SEQ ID NO: 59 - protein, 122 aa, synthetic construct
  REGION 1..122: note = VH2 of antibody PR001046 or PR003178, Chothia definition
EVQLVETGGGLIQPGGSLRLSCAASGFTVSDNYMTWVRQAPGKGLEWVSVIFSGGNTYYADSVKGRFTISRDNAKNTLYLQMNSLRAEDTALYYCARRNYDDTRGTDVFDIWGQGTMVTVSS

SEQ ID NO: 60 - protein, 122 aa, synthetic construct
  REGION 1..122: note = VH2 of antibody PR002299, Chothia definition
EVQLLESGGGLVQPGGSLRLSCAASGFTFSSYAMIWVRQAPGKGLEWVSGISESGGSTYYADSVKGRFTISRDNSKNTLYLQMNSLRAEDTAVYFCVKDLDDILTGYYKDYWGQGTLVTVSS

SEQ ID NO: 61 - protein, 123 aa, synthetic construct
  REGION 1..123: note = VH1 of antibody PR002199, Chothia definition
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSS

SEQ ID NO: 62 - protein, 125 aa, synthetic construct
  REGION 1..125: note = VH1 of antibody PR002299, Chothia definition
EVQLLESGGGLVQPGGSLRLSCAASGFTFSTYAMNWVRQAPGKGLEWVSRIRSKYNNYATYYADSVKDRFTISRDDSKSTLYLQMNSLRAEDTAVYYCVRHGNFGNSYVSWFAYWGQGTLVTVSS

SEQ ID NO: 63 - protein, 125 aa, synthetic construct
  REGION 1..125: note = VH1 of antibody PR003178, Chothia definition
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSS

SEQ ID NO: 64 - protein, 107 aa, synthetic construct
  REGION 1..107: note = VL of antibody PR000274, Chothia definition
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIK

SEQ ID NO: 65 - protein, 107 aa, synthetic construct
  REGION 1..107: note = VL of antibody PR002199, Chothia definition
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIK

SEQ ID NO: 66 - protein, 109 aa, synthetic construct
  REGION 1..109: note = VL of antibody PR002299 or antibody PR003178, Chothia definition
QAVVTQEPSLTVSPGGTVTLTCRSSTGAVTTSNYANWVQQKPGQAPRGLIGGTNKRAPWTPARFSGSLLGDKAALTLLGAQPEDEAEYFCALWYSNLWVFGGGTKLTVL

SEQ ID NO: 67 - protein, 5 aa, synthetic construct
GGGGS

SEQ ID NO: 68 - protein, 15 aa, synthetic construct
GGGGSGGGGSGGGGS

SEQ ID NO: 69 - protein, 20 aa, synthetic construct
GGGGSGGGGSGGGGSGGGGS

(19) United States
(12) Patent Application Publication
(10) Pub. No.: US 2026/0062493 A1
(43) Pub. Date: Mar. 5, 2026

(54) Antibody Targeting BCMA, Bispecific Antibody, And Use Thereof

(71) Applicant: HARBOUR BIOMED (SHANGHAI) CO., LTD., Shanghai (CN)

(72) Inventors: Lei SHI, Shanghai (CN); Xuekun ZHANG, Suzhou (CN); Yun HE, Shanghai (CN); Jianxun ZHAO, Suzhou (CN); Wei ZHOU, Suzhou (CN); Fei CHEN, Suzhou (CN); Xiangyang TAN, Suzhou (CN); Yiping RONG, Shanghai (CN)

(21) Appl. No.: 19/348,232

(22) Filed: Oct. 2, 2025

Related U.S. Application Data
(63) Continuation of application No. 17/764,308.

(30) Foreign Application Priority Data
Sep. 30, 2019 (CN) 201910941311.0

(51) Int. Cl. C07K 16/28; A61K 39/00; A61K 47/68; A61P 35/00

(52) U.S. Cl. CPC C07K 16/2878; A61K 47/6849; A61P 35/00; C07K 16/2809; A61K 2039/505; C07K 2317/21; C07K 2317/24; C07K 2317/31; C07K 2317/53; C07K 2317/565; C07K 2317/732; C07K 2317/734; C07K 2317/76; C07K 2317/92

(57) ABSTRACT

Provided are an antibody targeting BCMA, a bispecific antibody targeting BCMA and CD3, and applications thereof. The antibody targeting BCMA comprises two heavy chain variable regions, the heavy chain variable regions comprising HCDR1, HCDR2, and HCDR3 as shown in the amino acid sequences of SEQ ID NO: 22, SEQ ID NO: 33 and SEQ ID NO: 42, or HCDR1, HCDR2, and HCDR3 as shown in the amino acid sequences of SEQ ID NO: 26, SEQ ID NO: 37 and SEQ ID NO: 41. The antibody effectively targets human BCMA and binds to cynomolgus BCMA; the bispecific antibody effectively targets both human BCMA and human CD3, and can bind to cynomolgus BCMA and cynomolgus CD3.

### CROSS-REFERENCE TO RELATED APPLICATIONS

This application is a continuation application of U.S. patent application Ser. No. 17/764,308, filed on Mar. 28, 2022, which is the U.S. National Stage of International Application No. PCT/CN2020/118780, filed on Sep. 29, 2020, which claims priority to Chinese patent application 201910941311.0, filed on Sep. 30, 2019. The present application incorporates by reference the above-mentioned Chinese patent applications in their entirety.

### REFERENCE TO SEQUENCE LISTING

This application contains references to nucleic acid sequences and/or amino acid sequences which have been submitted concurrently herewith as the sequence listing XML file entitled “17424-000114-US-COA_11_Sept_2025_ST26.xml”, file size 93,684 Bytes (B), created on 11 Sep. 2025. The aforementioned sequence listing is hereby incorporated by reference in its entirety.

### TECHNICAL FIELD

The present disclosure belongs to the field of biopharmaceuticals, and particularly relates to an antibody targeting BCMA, bispecific antibody and use thereof.

### BACKGROUND

BCMA (B-cell maturation antigen, TNFRSF17, CD269) is a transmembrane protein belonging to the TNF receptor superfamily. As a non-glycosylated transmembrane protein, BCMA involved in B-cell maturation, growth and survival. BCMA is the receptor of two ligands of TNF superfamily: high affinity ligand APRIL (proliferation inducing ligand) and low affinity ligand BAFF (B cell activating factor). As a highly differentiated plasma cell-selective protein, the expression of BCMA is restricted to B-cell lineage, and is found mainly on plasma cells and plasma mother cells, and memory B-cells to a certain extent, but not on peripheral B-cells. BCMA is expressed in malignant plasma cells from patients with multiple myeloma (MM), supporting the growth and survival of multiple myeloma cells.

Multiple myeloma is the second most hematologic malignancy after non-Hodgkin's lymphoma, accounting for about 1% of all malignancies, 13% of hematologic malignancies, and 2% of deaths due to malignancies. Myeloma cells usually proliferate clonally in the bone marrow and the soft tissues of skeletal sponges, causing osteolytic bone destruction and poor healing mostly associated with anemia, renal failure and multiple damages caused by extramedullary infiltration of myeloma cells. As of 2006, the 5 annual relative productivity of MM is approximately 34%, and all therapies currently used for MM are non-curative. As an emerging target in multiple myeloma, BCMA antibodies can act on MM cells through multiple mechanisms. The research and development involving BCMA has focused on monoclonal antibodies, CAR-T therapies, ADCs, and bispecific antibody therapies.

The prior art antibody1 (hereinafter referred to as the positive control, and numbered PR000274 in the Examples) is the anti-BCMA antibody CA8-J6MO hIgG1 from GlaxoSmithKline (GSK). At present, the antibody conjugated drug bellantamab mafodotin (CA8-J6M0-mcMMAF, GSK2857916) prepared by GSK based on the anti-BCMA antibody CA8-J6MO has been used to treat different types of MM patients in a number of clinical trials. The sequence of this control antibody was derived from (Tabs-therapeutic Antibody database). Data from a small clinical study showed that the objective response rate (ORR) was 60% and the median progression free survival (PFS) was 12 months in 35 patients with over-pretreated (most patients had failed at least five therapy) relapsed or refractory (R/R) multiple myeloma (MM). In terms of safety, the most common side effects include corneal events, thrombocytopenia and anemia, all of which are associated with cytotoxic agents conjugated in ADC.

Currently, bispecific antibodies in clinical development include AMG-420 from Amgen, REGN-5458 from Regeneron, CC-93269 from Shinkee, JNJ-64007957 from Johnson & Johnson, and TNB383B from AbbVie. However, these bispecific antibodies also suffer from problems such as short half-lives and cytokine release syndrome (CRS). The prior art antibody 2 (hereinafter referred to as the positive control 2, and numbered PR002199 in the Examples) is an anti-BCMA (TNB308902) ×CD3 (TNB_F2B) bispecific antibody deriving from Teneobio's patent WO2018052503. Teneobio's TNB383B bispecific antibody for MM has entered clinical phase I in the 2019. The TNB383B uses a weakened CD3 antibody with attenuated cytokine release. However, TNB383B does not bind to CD3 and BCMA in cynomolgus monkeys and cannot be evaluated toxicologically in cynomolgus monkeys.

Therefore, there is an urgent need to develop safer and more effective monoclonal antibodies targeting BCMA and binding to BCMA of cynomolgus monkeys, as well as safer and more effective bispecific antibodies targeting both human BCMA and CD3 and binding to BCMA and CD3 of cynomolgus monkeys.

### SUMMARY

In order to solve the technical problems of the lack of safe and effective monoclonal antibodies targeting human BCMA and binding BCMA of cynomolgus monkeys, and the defect of bispecific antibodies targeting both human BCMA and CD3and binding BCMA and CD3 of cynomolgus monkeys in the prior art, the present disclosure provides an antibody targeting BCMA, bispecific antibodies targeting BCMA and CD3, and uses thereof.

To solve the above-mentioned technical problems, the technical solution of the first aspect in the present disclosure is: an antibody targeting BCMA comprising two heavy chain variable regions, wherein the heavy chain variable region comprises HCDR1, HCDR2 and HCDR3 as shown in the amino acid sequences of SEQ ID NO: 22, SEQ ID NO: 33 and SEQ ID NO: 42, respectively; or, comprises HCDR1, HCDR2 and HCDR3 as shown in the amino acid sequences of SEQ ID NO: 26, SEQ ID NO: 37 and SEQ ID NO: 41, respectively. The BCMA antibody of the present disclosure has binding activity to human BMCA and cynomolgus monkey BCMA. The antibody is only half the size of conventional IgG antibodies, which can be used in developing bispecific antibody and solving the problems of light chain mismatch and heterodimerization.

In a preferred specific embodiment, the heavy chain variable region has amino acid sequences of SEQ ID NO: 59; or the heavy chain variable region has amino acid sequences of SEQ ID NO: 60 or mutant thereof; the mutant has an addition, substitution or deletion of one or more amino acids on the original amino acid sequence, maintaining or improving the binding capacity of the first protein functional region to the BCMA antigen. Preferably, the mutant has 80%, 85%, 90%, 95%, 96%, 97%, 98%, 99% or more of sequence identity to the amino acid sequence as shown in SEQ ID NO: 59, SEQ ID NO: 60.

In a preferred specific embodiment, the antibody targeting BCMA further comprises an Fc fragment. Thereby, the antibody targeting BCMA comprises two heavy chains. Preferably, the Fc fragment is an Fc fragment of hIgG1, hIgG2, hIgG3, hIgG4 or mutation thereof.

In a preferred specific embodiment, the heavy chain has an amino acid sequence of SEQ ID NO: 1, SEQ ID NO: 7 or SEQ ID NO: 8.

To solve the above-mentioned technical problem, the technical solution of the second aspect in the present disclosure is: a bispecific antibody, comprising a first protein functional region targeting BCMA and a second protein functional region targeting CD3, wherein, the first protein functional region comprises the heavy chain variable region of the antibody targeting BCMA of the technical solution in the first aspect; preferably, the two heavy chain variable regions are linked by (G₄S)ₙ, the n is a non-0 natural number, preferably 1-20; more preferably 3 or 4. The BCMA×CD3 bispecific antibody of the present disclosure is a unique BCMA×CD3 bispecific antibody with two sites for binding to BCMA, thereby improving the affinity and specificity of binding to BCMA on tumor cells; both protein functional regions of the bispecific antibody have good binding activity to cynomolgus monkey. The sequence G₄S is numbered as SEQ ID NO: 67. When n is 3, the sequence (G₄S)ₙ is numbered as SEQ ID NO: 68. When n is 4, the sequence (G₄S)ₙ is numbered as SEQ ID NO: 69.

In a preferred specific embodiment, the second protein functional region comprises a heavy chain variable region and a light chain variable region, the heavy chain variable region has HCDR1, HCDR2 and HCDR3 with amino acid sequences of SEQ ID NO: 25, SEQ ID NO: 38 and SEQ ID NO: 44, respectively, and/or, the light chain variable region has LCDR1, LCDR2 and LCDR3 with amino acid sequences of SEQ ID NO: 50, SEQ ID NO: 53 and SEQ ID NO: 56, respectively.

Preferably, the heavy chain variable region of the second protein functional region has an amino acid sequence of SEQ ID NO: 63, SEQ ID NO: 62 or mutant thereof, and the light chain variable region has an amino acid sequence of SEQ ID NO: 66 or mutant thereof, and the mutant maintains or improves the binding of the second protein functional region to CD3. The bispecific antibody of the present disclosure optimizes the activity of the CD3 terminus, reduces the release of cytokines and reduces the toxicity of the BCMA×CD3 bispecific antibody.

In a preferred specific embodiment, the bispecific antibody further comprises a heavy chain constant region and a light chain constant region, preferably a human heavy chain constant region and a human light chain constant region, and the human light chain constant region is preferably a human λ or κ light chain constant region, and the human heavy chain constant region is preferably a heavy chain constant region of hIgG1, hIgG2, hIgG3 or hIgG4 or mutant thereof.

Preferably, the mutant of the human heavy chain constant region is selected from the group consisting of:
(1) L234A/L235A mutation;
(2) knob mutation T366W and hole mutations T366S, L368A and Y407V;
(3) knob mutation S354C and hole mutation Y349C;

More preferably, the first protein functional region comprises a heavy chain with an amino acid sequence of SEQ ID NO: 8 or SEQ ID NO: 7, and the second protein functional region comprises a heavy chain with an amino acid sequence of SEQ ID NO: 5 or SEQ ID NO: 4 and a light chain with an amino acid sequence of SEQ ID NO: 47. The bispecific antibody of the present disclosure has a human Fc fragment that retains the binding effect on FcRn and thus has a long half-life.

To solve the above-mentioned technical problem, the technical solution of the third aspect in the present disclosure is to provide an isolated nucleic acids encoding the antibody targeting BCMA according to the first aspect of the present disclosure or the bispecific antibody as described in the second aspect of the present disclosure.

To solve the above-mentioned technical problem, the technical solution of the fourth aspect in the present disclosure is to provide an expression vector comprising the isolated nucleic acids according to the third aspect of the present disclosure.

To solve the above-mentioned technical problem, the technical solution of the fifth aspect in the present disclosure is to provide a host cell comprising an expression vector of the fourth aspect of the present disclosure; preferably, the host cell is a prokaryotic cell or a eukaryotic cell.

To solve the above-mentioned technical problem, the technical solution of the sixth aspect in the present disclosure is to provide antibody drug conjugate comprising the antibody targeting BCMA of the first aspect of the present disclosure or a bispecific antibody of the second aspect of the present disclosure, and a cytotoxic agent.

To solve the above-mentioned technical problem, the technical solution of the seventh aspect in the present disclosure is to provide a pharmaceutical composition comprising the antibody targeting BCMA of the first aspect of the present disclosure, the bispecific antibody of the second aspect of the present disclosure, or the antibody drug conjugate of the sixth aspect of the present disclosure, and a pharmaceutically acceptable carrier.

To solve the above-mentioned technical problem, the technical solution of the eighth aspect in the present disclosure is to provide a use of the antibody targeting BCMA of the first aspect of the present disclosure, the bispecific antibody of the second aspect of the present disclosure, the antibody drug conjugate of the sixth aspect of the present disclosure, or a pharmaceutical composition of the seventh aspect of the present disclosure in the preparing a medicament for treating and/or preventing cancer.

Furthermore, in order to solve the above-mentioned technical problems, the technical solution of the ninth aspect of the present disclosure provides a kit combination comprising kit A and kit B; the kit A comprises the antibody targeting BCMA of the first aspect in the present disclosure, the bispecific antibody of the second aspect in the present disclosure, the host cell of the fifth aspect in the present disclosure, the antibody drug conjugate of the sixth aspect in the present disclosure, or the pharmaceutical composition of the seventh aspect in the present disclosure; kit B comprises other antibodies, bispecific antibodies, host cells or pharmaceutical compositions, and the other antibodies, bispecific antibodies, host cells or pharmaceutical compositions targeting CD3, BCMA or other targets. Kit A and kit B can be used with no particular order, or kit A is used before kit B, or kit B is used before kit A.

The antibody targeting BCMA of the technical solution in the first aspect of the present disclosure, the bispecific antibody of the technical solution in the second aspect of the present disclosure, the host cell of the technical solution in the fifth aspect of the present disclosure, the antibody drug conjugate of the technical solution in the sixth aspect of the present disclosure, or the pharmaceutical composition of the technical solution in the seventh aspect of the present disclosure may be administered to a patient for the treatment of a relevant tumor.

On the basis of common knowledge in the art, each of the above preferred conditions can be combined in any way to obtain each preferred example of the present disclosure.

The reagents and raw materials used in the present disclosure are all commercially available.

The positive and progressive effects of the present disclosure are:
1. The BCMA antibody in the present disclosure is a novel full human antibody comprising only “heavy chain” with binding activity to human BMCA and cynomolgus monkey BCMA. The BCMA heavy chain antibody is only half the size of conventional IgG antibodies. The absence of a light chain in the antibody makes it possible to use in preparing bispecific antibodies and to solve the problems of light chain mismatch and heterodimerization.
2. The BCMA×CD3 bispecific antibody in the present disclosure: a. is a unique BCMA×CD3 bispecific antibody with two sites to bind BCMA, thus improves the affinity and specificity of binding to BCMA on tumor cells; b. has a bispecific antibody structure with a human Fc fragment that retains the binding effect of Fc to FcRn, thus having a long half-life; c. optimizes the activity of the CD3 terminus, reduces cytokine release, and decreases the toxicity of the BCMA×CD3 bispecific antibody; d. the BCMA terminus and CD3 terminus of the antibody have good binding activity to that of cynomolgus monkey.

### BRIEF DESCRIPTION OF THE DRAWINGS

FIG. 1 shows the binding capacity assay of anti-human BCMA HCAb monoclonal antibody at cellular level with FACS.

FIG. 2 shows the blockage assay of anti-human BCMA HCAb monoclonal antibody to the binding of human BCMA with ligand BAFF protein with ELISA.

FIG. 3 shows in vitro binding capacity assay of BCMA×CD3 bispecific antibody on NCI-H929 cell line with highly expressed BCMA with FACS.

FIG. 4 shows in vitro binding capacity assay of BCMA×CD3 bispecific antibody on HEK293T cell strain overexpressing human and cynomolgus monkey BCMA with FACS.

FIG. 5 shows in vitro binding capacity assay of BCMA×CD3 bispecific antibody on human and cynomolgus monkey T cells with FACS.

FIG. 6 shows in vitro killing experiment and cytokine release of BCMA×CD3 bispecific antibody against cell line NCI-H929 highly expressing BCMA.

FIG. 7 shows in vitro killing experiment and cytokine release of BCMA×CD3 bispecific antibody against cell line RPMI8226 low expressing BCMA.

FIG. 8 shows the binding of BCMA×CD3 bispecific antibody to BCMA-negative cell line HL-60 with FACS.

FIG. 9 shows detection of non-specific killing and cytokine release of BCMA×CD3 bispecific antibody against BCMA-negative cell line HL60 with FACS.

FIG. 10 shows evaluation of antitumor efficacy of BCMA×CD3 bispecific antibodies in NCI-H929/human PBMC mice model

FIG. 11 shows detection for affinity of PR003178 to human CD3e/g recombinant protein, cynomolgus monkey CD3e/g recombinant protein, human BCMA recombinant protein and cynomolgus monkey BCMA recombinant protein with BIACORE.

### DETAILED DESCRIPTION OF THE PREFERRED EMBODIMENT

The present disclosure will be further illustrated by the following examples but the present disclosure is not limited to the scope of examples thereto. The experimental methods for which specific conditions are not indicated in the following examples shall be selected according to conventional methods and conditions, or according to the specification of commodity.

In this application, the term “antibody” generally refers to a protein comprising an antigen-binding portion, and optionally a scaffold or backbone portion that allows the antigen-binding portion to apply a conformation that facilitates the binding of an antibody to an antigen. An antibody may typically comprise an antibody light chain variable region (VL), an antibody heavy chain variable region (VH), or both. For example, “heavy chain antibody” in the present application comprises a VH region without a VL region. A VH or VL region may be further divided into a hypervariable region called complementarity determining region (CDR), and a more conserved region called framework region (FR) where the CDRs are scattered. Each VH or VL may consist of three CDRs and four FRs, which can be arranged in the following order from amino terminus to carboxyl terminus: FR1, CDR1, FR2, CDR2, FR3, CDR3 and FR4. The variable regions of a heavy chain and a light chain comprise binding domains that interact with an antigen. The examples of an antibody include, but not limited to, full-length antibody, heavy chain antibody (HCAb), antigen-binding fragment (Fab, Fab′, F(ab)2, Fv fragment, F(ab′)2, scFv, di-scFv and/or dAb), immuno-conjugate, multispecific antibody (e.g., bispecific antibody), antibody fragment, antibody derivative, antibody analog, or fusion protein, etc, as long as they show the desired antigen-binding activity.

In this application, the term “variable” generally refers to the fact that certain portions of the sequence of a variable domain in an antibody varies intensively, which forms the binding and specificity of various specific antibodies to their specific antigen. However, the variability is not distributed uniformly in the entire variable region of an antibody. The variability is centralized in three segments of the variable regions of a light chain and a heavy chain, called CDR or hypervariable region (HVR), with a FR being the more highly-conserved portions of the variable domain. Each of the variable domains of natural heavy chain and light chains comprises four FRs, most of which are using β-fold conformation and linked by three CDRs, forming a loop linkage and being part of a β-fold structure in some cases. The CDRs in each chain are closely adjacent to each other through the FRs and are forming an antigen-binding site of an antibody together with the CDRs from the other chain. The constant regions are not directly involved in antibody-antigen binding, but they exhibit different effector functions, such as participating in antibody-dependent cytotoxicity of the antibody.

In the present art, the CDR of an antibody can be defined by various methods, such as Kabat definition rule (see Kabat et al., Sequences of Proteins of Immunological Interest, Fifth Edition, National Institutes of Health, Bethesda, MD (1991)) based on sequence variability and Chothia definition rule based on the regional position of a structural loop (see A1-Lazikani et al., JMol Biol 273:927-48, 1997). In the present application, Combined definition rule incorporating Kabat definition and Chothia definition was also used to determine amino acid residues in the sequences of a variable domain and a full-length antibody (Table1).

TABLE 1
Definition method for the CDRs of the antibody in the present
application (available at http://bioinf.org.uk/abs/)

| CDR region | Kabat definition | Chothia definition | Combined definition |
|---|---|---|---|
| LCDR1 | L24 - - - L34 | L24 - - - L34 | L24 - - - L34 |
| LCDR2 | L50 - - - L56 | L50 - - - L56 | L50 - - - L56 |
| LCDR3 | L89 - - - L97 | L89 - - - L97 | L89 - - - L97 |
| HCDR1 | H31 - - - H35 | H26 - - - H32 | H26 - - - H35 |
| HCDR2 | H50 - - - H65 | H52 - - - H56 | H50 - - - H65 |
| HCDR3 | H95 - - - H102 | H95 - - - H102 | H95 - - - H102 |

Wherein, Laa-Lbb can refer to the amino acid sequence from aa position (Chothia numbering rule) to bb position (Chothia numbering rule) started from N-terminal of the light chain of an antibody; Haa-Hbb can refer to the amino acid sequence from aa position (Chothia numbering rule) to bb position (Chothia numbering rule) started from N-terminal of the heavy chain of an antibody. For example, L24-L34 can refer to the amino acid sequence from position 24 to position 34 started from N-terminal of the light chain of an antibody according to Chothia numbering rule; H26-H32 can refer to the amino acid sequence from position 26 to position 32 started from N-terminal of the heavy chain of an antibody according to Chothia numbering rule.

The effector functions such as ADCC and CDC mediated by Fc domain of an antibody have significant biological functions, and different IgG isoforms have different ADCC or CDC functions. For example, IgG1 and IgG3 have relatively strong ADCC and CDC effects, while IgG2 and IgG4 have relatively weak effects. In addition, the original effector function of Fc can also be modulated by amino acid mutations or modifications to alter the binding capacity of Fc to the Fc receptor. For example, “LALA” double mutant (L234A/L235A) in IgG1 can significantly decrease its affinity to FcγRIIIA (CD16A) and thus decreases the ADCC effect. In addition, P329G mutation can significantly decrease the binding to multiple Fcγ receptors (see Schlothauer T, Herter S, Koller C F, Grau-Richards S, Steinhart V, Spick C, Kubbies M, Klein C, Umaña P, Mössner E. Novel human IgG1 and IgG4 Fc-engineered antibodies with completely abolished immune effector functions. Protein EngDes Sel. 2016 October; 29(10):457-466. doi: 10.1093/protein/gzw040. Epub 2016 Aug. 29. PubMed PMID: 27578889). In the application, in order to decrease the binding of BCMA×CD3 bispecific antibodies to Fcγ receptors, “LALA” double mutant (L234A/L235A) or “LALAPG” triple mutant (L234A/L235A/P329G) was introduced to the Fc of these antibodies.

### EXAMPLE 1. MOUSE IMMUNIZATION AND OBTAINING ANTI-BCMA ANTIBODY MOLECULES

BCMA antigens can be used to immunize experimental animals to obtain antibody molecules specifically binding to BCMA, and the experimental animals can be mice, rats, rabbits, sheep, camels, etc. Generally, the antibody molecules obtained are non-human origin. After obtaining non-human antibodies, these molecules need to be humanized using antibody engineering techniques to reduce immunogenicity and improve drug-forming properties. However, the humanization process of antibodies is complicated technically as the humanized molecules often have decreased affinity to the antigen. On the other hand, advances in transgenic technology have made it possible to breed genetically engineered mice carrying a human immunoglobulin immune repertoire without its endogenous murine immune repertoire. The antibody produced by this transgenic mouse has a full human sequence, so there is no need for further humanization, which greatly improves the development efficiency of therapeutic antibody. Harbour HCAb mice (Harbour Antibodies BV, WO 2002/085945 A3) are transgenic mice that carry a human immunoglobulin immune repertoire, and are capable of producing a novel only “heavy chain” antibody that is half the size of conventional IgG antibodies. The antibodies produced therefrom Harbour HCAb mice only have a “heavy chain” variable domain of a human antibody and a murine Fc constant domain. The absence of a light chain in this antibody almost makes it a solution to problems of light chain mismatch and heterodimerization, allowing this technology platform to develop products that are difficult to achieve with traditional antibody platforms.

### 1.1 Immunization of Mice with BCMA Antigen

Harbour HCAb mice were immunized with multiple rounds of a soluble recombinant human BCMA-ECD-Fc fusion protein. The antigen protein was mixed with an immune adjuvant to form an immunogenic reagent, which is then injected subcutaneously through groin or intraperitoneally. In each round of immunization, each mouse received a total injection dose of 100 μL. In the first round of immunization, each mouse received an immunization with an immunogenic reagent prepared by mixing 50 μg of antigen protein (recombinant human BCMA-ECD-Fc, ACRO, Cat. BC7-H82F0) and complete Freund's adjuvant (Sigma, #F5881) at a volume ratio of 1:1. In each subsequent booster of immunization, each mouse received an immunization with an immunogenic reagent prepared by mixing 25 μg of antigen protein and Sigma Adjuvant System adjuvant (Sigma, #S6322). The interval between each round of booster immunization was at least two weeks, and usually the immunization had no more than five rounds of booster immunization. The immunization time was day 0, 14, 28, 42, 56 and 70 the murine serum antibody titers were detected on days 49 and 77. The final booster immunization was performed at a dosage of 25 μg of antigen protein per mouse five days before the isolation of spleen B cells from HCAb mice.

### 1.2 Obtaining HCAb Monoclonal and Antibody Sequences

When the murine serum antibody titer specific to BCMA reached a certain level, murine spleen cells were removed to isolate B cells, and CD138-positive plasma cells and BCMA antigen-positive B cell populations were sorted by BD FACS AriaII Cell Sorter. RNA was extracted, and reverse transcripted to cDNA, then the human VH gene was amplified by PCR. The amplified VH gene fragment was constructed into the mammalian cell expression plasmid pCAG vector encoding the Fc domain sequence in the heavy chain of a human IgG1 antibody. The plasmid was transfected into mammalian host cells (e.g. human embryonic kidney cell HEK293) for expression. The antibody supernatant of HCAb expressed was screened by Mirrorball with recombinant human BCMA-Fc and Avitag recombinant protein (ACRO, Cat. Bc7-H82F0). The binding capacity of the Mirrorball-positive monoclonal antibodies obtained to cells of HEK293T cell strain overexpressing human BCMA (HEK293T/huBCMA, Beijing KangYuan), HEK293T cell strain overexpressing cynomolgus monkey BCMA (HEK293T/cynoBCMA, Beijing KangYuan) and NCI-H929 cell line highly expressing human BCMA (ATCC® CRL-9068™) were further identified by FACS. The clone number of the positive clone obtained by screening is HBM1005P63B7, hereafter numbered PR001046. The nucleotide sequence encoding the variable domain of the antibody molecule and corresponding amino acid sequence were obtained by using conventional sequencing methods. In the present example, the sequence of the variable domain of the anti-BCMA monoclonal antibody molecule obtained from immunized Harbour HCAb mice was a human antibody sequence.

Table 2 lists the amino acid sequences of the variable domain and full length of the heavy chain, and the amino acid sequences of the CDRs defined according to Combined definition rule of the BCMA antibody in this example. See the sequencing list for the amino acid sequences of the CDRs defined according to other rules for the antibodies of the present disclosure.

TABLE 2
Amino acid sequences associated with anti-BCMA HCAb antibodies

| PR001046 | Amino acid sequence and sequence id number |
|---|---|
| Heavy chain sequence | EVQLVETGGGLIQPGGSLRLSCAASGFTVSDNYMTWVRQ |
| of PR001046 | APGKGLEWVSVIFSGGNTYYADSVKGRFTISRDNAKNTLY |
| | LQMNSLRAEDTALYYCARRNYDDTRGTDVFDIWGQGTM |
| | VTVSSEPKSCDKTHTCPPCPAPELLGGPSVFLFPPKPKDTL |
| | MISRTPEVTCVVVDVSHEDPEVKFNWYVDGVEVHNAKTK |
| | PREEQYNSTYRVVSVLTVLHQDWLNGKEYKCKVSNKALP |
| | APIEKTISKAKGQPREPQVYTLPPSREEMTKNQVSLTCLVK |
| | GFYPSDIAVEWESNGQPENNYKTTPPVLDSDGSFFLYSKLT |
| | VDKSRWQQGNVFSCSVMHEALHNHYTQKSLSLSPGK |
| | (SEQ ID NO: 1) |
| Heavy chain variable | EVQLVETGGGLIQPGGSLRLSCAASGFTVSDNYMTWVRQ |
| region sequence of | APGKGLEWVSVIFSGGNTYYADSVKGRFTISRDNAKNTLY |
| PR001046 | LQMNSLRAEDTALYYCARRNYDDTRGTDVFDIWGQGTM |
| | VTVSS (SEQ ID NO: 59) |
| PR001046 VH CDR1 | GFTVSDNYMT (SEQ ID NO: 22) |
| (Combined) | |
| PR001046 VH CDR2 | VIFSGGNTYYADSVKG (SEQ ID NO: 33) |
| (Combined) | |
| PR001046 VH CDR3 | RNYDDTRGTDVFDI (SEQ ID NO: 42) |
| (Combined) | |

### 1.3 Preparation of Anti-BCMA Full Human Recombinant Antibody

Plasmids encoding the heavy chain of antibody PR001046 were transfected into mammalian host cells (e.g. human embryonic kidney cell HEK293) to obtain purified anti-BCMA recombinant heavy chain antibodies using conventional recombinant protein expression and purification techniques. Specifically, HEK293 cells were cultured for expansion in FreeStyle™ F17 Expression Medium (Thermo, A1383504). Prior to starting transient transfection, the cell concentration was adjusted to 6×10⁵ cells/ml. The cells were incubated at 37° C., 8% CO₂ in a shaker for 24 hours until the cell concentration reached 1.2×10⁶ cells/ml. 30 ml of cultured cells was prepared, and then 30 μg of plasmids encoding PR001046 heavy chain described above was dissolved in 1.5 ml Opti-MEM reduced serum medium (Thermo, 31985088), and then 1.5 ml of Opti-MEM was taken to dissolve 120 μl of 1 mg/ml PEI (Polysciences, Inc, Cat #23966-2) and leave aside for 5 min. PEI was added slowly to the plasmids and incubated for 10 min at room temperature. The mixture of PEI and plasmids was dropped slowly into the cells with the culture flask shaking, and the cells were incubated at 37° C., 8% CO₂ in a shaker for 5 days. Cell viability was observed after 5 days. The cultures were collected and centrifuged at 3300 G for 10 minutes for the supernatant; then the supernatant was subject to high speed centrifugation to remove impurities. Gravity columns (Bio-Rad, #7311550) containing MabSelect® (GE Healthcare Life Science, Cat #71-5020-91 AE) were equilibrated with PBS (pH 7.4) and washed with 2-5 folds of column volume. The supernatant sample was subject to column chromatography. The columns were washed with 5-10 folds of column volume of PBS. The target protein was then eluted with 0.1 M glycine (pH 3.5), and adjusted to neutral with Tris-HCl (pH 8.0), and finally the solution was concentrated and exchanged with PBS buffer by concentration using ultrafiltration tubes (Millipore, UFC901024). The purified anti-BCMA heavy chain antibody solution was obtained.

The following table shows the sequences of light chain and heavy chain, variable region of the light chain and the heavy chain, and CDRs defined according to Combined definition rule of positive control 1, i.e., the full-length antibody PR000274.

TABLE 3
Amino acid sequences associated with full human recombinant
antibody

| PR000274 | Amino acid sequences and sequence id number |
|---|---|
| heavy chain sequence | QVQLVQSGAEVKKPGSSVKVSCKASGGTFSNYWMHWVRQ |
| of PR000274 | APGQGLEWMGATYRGHSDTYYNQKFKGRVTITADKSTSTA |
| | YMELSSLRSEDTAVYYCARGAIYDGYDVLDNWGQGTLVT |
| | VSSASTKGPSVFPLAPSSKSTSGGTAALGCLVKDYFPEPVTV |
| | SWNSGALTSGVHTFPAVLQSSGLYSLSSVVTVPSSSLGTQT |
| | YICNVNHKPSNTKVDKKVEPKSCDKTHTCPPCPAPELLGGP |
| | SVFLFPPKPKDTLMISRTPEVTCVVVDVSHEDPEVKFNWYV |
| | DGVEVHNAKTKPREEQYNSTYRVVSVLTVLHQDWLNGKE |
| | YKCKVSNKALPAPIEKTISKAKGQPREPQVYTLPPSREEMTK |
| | NQVSLTCLVKGFYPSDIAVEWESNGQPENNYKTTPPVLDSD |
| | GSFFLYSKLTVDKSRWQQGNVFSCSVMHEALHNHYTQKSL |
| | SLSPGK (SEQ ID NO: 2) |
| Heavy chain variable | QVQLVQSGAEVKKPGSSVKVSCKASGGTFSNYWMHWVRQ |
| region sequence of | APGQGLEWMGATYRGHSDTYYNQKFKGRVTITADKSTSTA |
| PR000274 | YMELSSLRSEDTAVYYCARGAIYDGYDVLDNWGQGTLVT |
| | VSS (SEQ ID NO: 58) |
| PR000274 VH CDR1 | GGTFSNYWMH (SEQ ID NO: 21) |
| (Combined) | |
| PR000274 VH CDR2 | ATYRGHSDTYYNQKFKG (SEQ ID NO:34) |
| (Combined) | |
| PR000274 VH CDR3 | GAIYDGYDVLDN (SEQ ID NO: 40) |
| (Combined) | |
| Light chain sequence | DIQMTQSPSSLSASVGDRVTITCSASQDISNYLNWYQQKPG |
| of PR000274 | KAPKLLIYYTSNLHSGVPSRFSGSGSGTDFTLTISSLQPEDFA |
| | TYYCQQYRKLPWTFGQGTKLEIKRTVAAPSVFIFPPSDEQL |
| | KSGTASVVCLLNNFYPREAKVQWKVDNALQSGNSQESVTE |
| | QDSKDSTYSLSSTLTLSKADYEKHKVYACEVTHQGLSSPVT |
| | KSFNRGEC (SEQ ID NO: 45) |
| Light Chain Variable | DIQMTQSPSSLSASVGDRVTITCSASQDISNYLNWYQQKPG |
| region Sequence of | KAPKLLIYYTSNLHSGVPSRFSGSGSGTDFTLTISSLQPEDFA |
| PR000274 | TYYCQQYRKLPWTFGQGTKLEIK (SEQ ID NO: 64) |
| PR000274 VL CDR1 | SASQDISNYLN (SEQ ID NO: 48) |
| (Combined) | |
| PR000274 VL CDR2 | YTSNLHS (SEQ ID NO: 51) |
| (Combined) | |
| PR000274 VL CDR3 | QQYRKLPWT (SEQ ID NO: 54) |
| (Combined) | |

### EXAMPLE 2. BINDING CAPACITY ASSAY OF ANTI-HUMAN BCMA HCAB Monoclonal Antibody At Cellular Level With FACS

This example was conducted to study the binding activity of anti human BCMA HCAb monoclonal antibodies to human and cynomolgus monkey BCMA in vitro. HEK293T cell strain (HEK293T/huBCMA, Beijing Kang yuan) overexpressing human BCMA, HEK293T cell strain (HEK293T/cynoBCMA, Beijing Kang yuan) overexpressing cynomolgus monkey BCMA and NCI-H929 cell line (ATCC® CRL-9068™) highly expressing human BCMA were used for antibody binding assay at cellular level. Briefly, HEK293T/huBCMA cells and HEK293T/cynoBCMA cells were digested and resuspended with DMEM complete medium, and NCI-H929 cell suspensions were collected by resuspension with DMEM complete medium. The density of the three cells was adjusted to 1×10⁶ cells/mL, respectively. The cells were inoculated at 100 μL cells/well in 96 well V-bottom plates (Corning, Cat #: 3894), followed by the addition of test antibodies at 100 μL/well, which is gradient diluted at 3 folds of the concentration with 2 folds of the final concentration. Cells were incubated at 4° C. for 1 hours in the dark. Afterwards, the cells were washed twice with 100 μL/well of pre-cooled PBS, centrifuged at 500 g for 5 min at 4° C. and followed by discarding the supernatant. Fluorescent secondary antibody (Alexa Fluor®) 488-conjugated AffiniPure Goat Anti-Human IgG, Fcγ Fragment Specific, Jackson, Cat #: 109-545-06,1:500 dilution) was added at 100 μL/well and incubated for 30 min at 4° C. in the dark. Cells were washed twice with 100 μL/well of pre-cooled PBS, centrifuged at 500 g, 4° C. for 5 min, and followed by discarding the supernatant. Finally, the cells were resuspended with 200 μL/well of pre-cooled PBS and the fluorescent luminescence signal values were read by using BD FACS CANTOII.

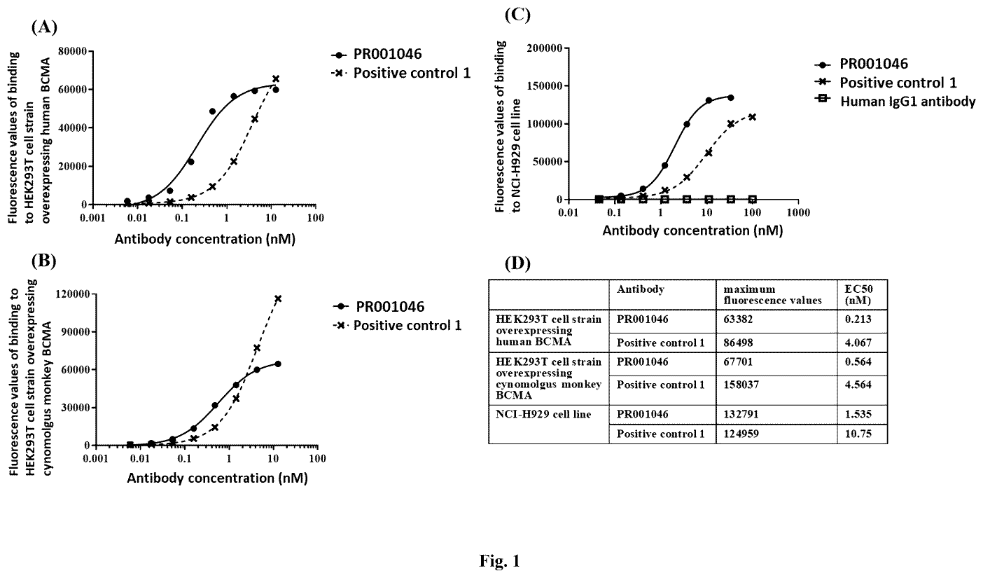

As shown in FIG. 1, the binding capacity of the anti-human BCMA HCAb monoclonal antibody PR001046 to the HEK293T cell strain overexpressing human BCMA (A), the HEK293T cell strain overexpressing cynomolgus monkey BCMA (B) and the cell line NCI-H929 (C) highly expressing human BCMA were all better than positive control 1 (PR000274). Specifically, the EC50 of the binding curve of PR001046 was lower than that of positive control 1, see (D) in the FIG. 1 for the specific maximum fluorescence values and EC50.

### EXAMPLE 3. BLOCKAGE ASSAY OF ANTI-HUMAN BCMA HCAB MONOCLONAL ANTIBODY TO THE BINDING OF HUMAN BCMA WITH LIGAND BAFF PROTEIN WITH ELISA

This example was conducted to evaluate the blockage ability of anti-human BCMA HCAb monoclonal antibody to the binding of human BCMA (ACRO, BCA-H522y-100 μg) with ligand BAFF protein (ACRO, BAF-H5248-50 μg). The BAFF protein was biotinylated by using a biotinylation kit (ThermoFisher, A39257, EZ-Link®) Sulfo-NHS-LC-Biotin) according to the instructions. The plates (Corning, Cat #: 9018) were coated with 1 μg/mL of human BCMA protein with Fc Tag (ACRO, Cat #: BC7-H5254) overnight. The plates were washed for 3 times with PBST, added with 2% BSA and then sealed at room temperature for 1 hour. The plates were washed with PBST for 3 times, and added with test antibody gradient diluted at 3 folds concentration 100 μL/well, with an initial concentration of 50 nM. After being incubated for 1 h, the supernatant was discarded and 0.5 μg/mL of biotinylated human BAFF protein (prepared according to conventional methods in the art) was added to the plates at 100 μL/well. The plates were incubated at room temperature for 1 h and discarded the supernatant. Streptavidin-coupled HRP (Bio-RAD, Cat #: 1610380) with 1:4000 diluted was added at 100 μL/well. The plates were incubated for 1 h at room temperature and washed with PBST for 3 times. TBM was added at 100 μL/well for color developing and the reaction was terminated 15 min later by adding termination solution. OD450 values were read by using Enspire (Perkin Elmer).

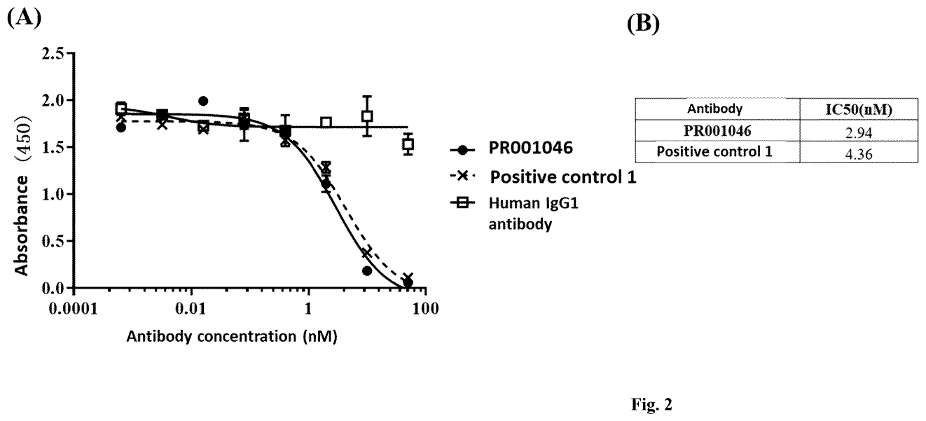

As shown in (A) of the FIG. 2, anti-human BCMA HCAb monoclonal antibody PR001046 has the ability to block the binding of human BCMA with its ligand BAFF protein comparable to that of positive control 1 antibody. The specific IC50 values is shown in the FIG. 2(B).

### EXAMPLE 4. HUMANIZATION OF CD3 ANTIBODY

Anti-CD3 antibody is an important component in the construction of T-Cell Engager Bispecific Antibody (T-Cell Engager Bispecific Antibody). Humanization was performed on the known murine antibody SP34 binding to human CD3e (CD3 epsilon single subunit or complex containing CD3c). As is described in WO2017084495A1 (Jiangsu Hengrui) or CN104136610B (Chugai Sangyo Co., Ltd.), the modification is briefly described as follows:

This example uses a “CDR transplantation” method for sequence humanization, that is, the VH CDRs of a murine antibody are transplanted to the VH framework region of a human antibody, and the VL CDRs of the murine antibody are transplanted to the VL framework region of the human antibody. The sequence of the framework region in VH or VL of human antibody can be derived from human germline gene sequence, or antibody sequences after V (D) J rearrangement, or the consensus sequence of a specific VH or VL gene family of human antibody. In this example, the frame region sequence provided by the human germline gene sequence was used as the humanized template sequence, that is, the human germline V gene fragment provides the sequence of frame regions FR1, FR2 and FR3, and the human germline J gene fragment provides the sequence of frame region FR4. Finally, humanized variable region (VH or VL) sequences were constructed in the order of (human) FR1-(murine) CDR1-(human) FR2-(murine) CDR2-(human) FR3-(murine) CDR3-(human) FR4.

In this example, the sequence of human germline V gene fragment IGHV3-73*01 or human germline V gene fragment IGHV3-23*01 combined with human germline J gene fragment IGHJ1*01 is used as a humanized template to provide a frame region sequence. And amino acid mutation at one or more sites was introduced at position 30, 73, 76, 78, 93 or 94 (according to Chothia numbering rules) to obtain various different VH variant sequences.

In this example, the sequence of the human germline V gene fragment IGLV7-46*02 combined with the sequence of the human germline J gene fragment IGLJ2*01or the sequence of the human germline V gene fragment IGKV1-39*01 combined with the sequence of the human germline J gene fragment IGKJ4*01 is used as a humanized template to provide the frame region sequence. And amino acid mutation at none or more sites was introduced at position 2, 36, 46, 49, 66, 69, 71 or 87 (according to Chothia numbering rules) to obtain various different VL variant sequences. The following table 4 shows amino acid sequence of one of the CD3 antibodies, the sequences of the light chain (LC) and the heavy chain (hereinafter, the heavy chain and heavy chain variable region of the CD3 antibody of the present disclosure are referred to as HC1 and VH1 in the bispecific antibody, respectively) of this antibody and the heavy chain (hereinafter, the heavy chain and heavy chain variable region of the BCMA antibody of the present disclosure are referred to as HC2 and VH2 in the bispecific antibody, respectively) sequence of the BCMA antibody of the present disclosure will form the bispecific antibodies PR003178 and PR002299 (see Example 5 for the detailed construction method).

TABLE 4
Amino acid sequences of the obtained CD3 antibodies
Sequence information of CD 3antibody used for PR003178

| | |
|---|---|
| sequence of | EVQLVESGGGLVQPGGSLKLSCAASGFTFSTYAMNWVRQASGK |
| PR003178 | GLEWVGRIRSKYNNYATYYADSVKDRFTISRDDSKNTAYLQMNS |
| HC1 | LKTEDTAVYYCTRHGNFGNSYVSWFAYWGQGTLVTVSSASTKG |
| | PSVFPLAPSSKSTSGGTAALGCLVKDYFPEPVTVSWNSGALTSGV |
| | HTFPAVLQSSGLYSLSSVVTVPSSSLGTQTYICNVNHKPSNTKVDK |
| | KVEPKSCDKTHTCPPCPAPEAAGGPSVFLFPPKPKDTLMISRTPEV |
| | TCVVVDVSHEDPEVKFNWYVDGVEVHNAKTKPREEQYNSTYRV |
| | VSVLTVLHQDWLNGKEYKCKVSNKALPAPIEKTISKAKGQPREP |
| | QVYTLPPCREEMTKNQVSLWCLVKGFYPSDIAVEWESNGQPENN |
| | YKTTPPVLDSDGSFFLYSKLTVDKSRWQQGNVFSCSVMHEALHN |
| | HYTQKSLSLSPGK (SEQ ID NO: 5) |
| sequence of | EVQLVESGGGLVQPGGSLKLSCAASGFTFSTYAMNWVRQASGK |
| PR003178 | GLEWVGRIRSKYNNYATYYADSVKDRFTISRDDSKNTAYLQMNS |
| VH1 | LKTEDTAVYYCTRHGNFGNSYVSWFAYWGQGTLVTVSS (SEQ ID |
| | NO: 63) |
| sequence of | EVQLLESGGGLVQPGGSLRLSCAASGFTFSTYAMNWVRQAPGKG |
| PR002299 | LEWVSRIRSKYNNYATYYADSVKDRFTISRDDSKSTLYLQMNSLR |
| HC1 | AEDTAVYYCVRHGNFGNSYVSWFAYWGQGTLVTVSSASTKGPS |
| | VFPLAPSSKSTSGGTAALGCLVKDYFPEPVTVSWNSGALTSGVHT |
| | FPAVLQSSGLYSLSSVVTVPSSSLGTQTYICNVNHKPSNTKVDKK |
| | VEPKSCDKTHTCPPCPAPEAAGGPSVFLFPPKPKDTLMISRTPEVT |
| | CVVVDVSHEDPEVKFNWYVDGVEVHNAKTKPREEQYNSTYRVV |
| | SVLTVLHQDWLNGKEYKCKVSNKALGAPIEKTISKAKGQPREPQ |
| | VYTLPPCREEMTKNQVSLWCLVKGFYPSDIAVEWESNGQPENNY |
| | KTTPPVLDSDGSFFLYSKLTVDKSRWQQGNVFSCSVMHEALHNH |
| | YTQKSLSLSPGK (SEQ ID NO: 4) |
| sequence of | EVQLLESGGGLVQPGGSLRLSCAASGFTFSTYAMNWVRQAPGKG |
| PR002299 | LEWVSRIRSKYNNYATYYADSVKDRFTISRDDSKSTLYLQMNSLR |
| VH1 | AEDTAVYYCVRHGNFGNSYVSWFAYWGQGTLVTVSS (SEQ ID |
| | NO: 62) |
| PR003178/ | GFTFSTYAMN (SEQ ID NO: 25) |
| PR002299 | |
| VH1 CDR1 | |
| (Combined) | |
| PR003178/ | RIRSKYNNYATYYADSVKD (SEQ ID NO: 38) |
| PR002299 | |
| VH1 CDR2 | |
| (Combined) | |
| PR003178/ | HGNFGNSYVSWFAY (SEQ ID NO: 44) |
| PR002299 | |
| VH1 CDR3 | |
| (Combined) | |
| sequence of | QAVVTQEPSLTVSPGGTVTLTCRSSTGAVTTSNYANWVQQKPGQ |
| PR003178/ | APRGLIGGTNKRAPWTPARFSGSLLGDKAALTLLGAQPEDEAEYF |
| PR002299LC | CALWYSNLWVFGGGTKLTVLGQPKAAPSVTLFPPSSEELQANKA |
| | TLVCLISDFYPGAVTVAWKADSSPVKAGVETTTPSKQSNNKYAA |
| | SSYLSLTPEQWKSHRSYSCQVTHEGSTVEKTVAPTECS (SEQ ID |
| | NO: 47) |
| sequence of | QAVVTQEPSLTVSPGGTVTLTCRSSTGAVTTSNYANWVQQKPGQ |
| PR003178 VL | APRGLIGGTNKRAPWTPARFSGSLLGDKAALTLLGAQPEDEAEYF |
| | CALWYSNLWVFGGGTKLTVL (SEQ ID NO: 66) |
| PR003178/ | RSSTGAVTTSNYAN (SEQ ID NO: 50) |
| PR002299 | |
| LCDR1 | |
| (Combined) | |
| PR003178/ | GTNKRAP (SEQ ID NO: 53) |
| PR002299 | |
| LCDR2 | |
| (Combined) | |
| PR003178/ | ALWYSNLWV (SEQ ID NO: 56) |
| PR002299 | |
| LCDR3 | |
| (Combined) | |

### EXAMPLE 5. PREPARATION OF BCMA×CD3 BISPECIFIC ANTIBODY

The anti-BCMA antibody and anti-CD3 cells selected from Example 4 were used to prepare bispecific antibody. The prepared bispecific antibody of this BCMA×CD3 bispecific antibody can simultaneously bind two targets, wherein one terminus can recognize BCMA specifically expressed on the surface of a tumor cell, while the other terminus can bind to CD3 molecules on T cells. When the BCMA×CD3bispecific antibody molecule binds to the surface of a tumor cell, it can recruit and activate the T cells near the tumor cells, thus killing the tumor cells.

Each bispecific antibody involves three protein chains comprising the heavy chain of the corresponding anti-BCMA antibody, as well as the heavy chain and the light chain of the above-mentioned anti-CD3 antibody, respectively. To minimize the formation of by-products containing mismatched heavy chains (e.g. two heavy chains of the anti-CD3 antibody are mismatched), mutated heterodimeric Fc regions carrying “knob-hole” mutations and transformed disulfide bonds were used, as described in WO 2009080251 and WO 2009080252. The BCMA×CD3 bispecific antibody is IgG1with L234A and L235A mutations in Fc (according to EU index numbering).

Each bispecific antibody was generated by simultaneous co-transfection with three different mammalian expression vectors which encoding: 1) the heavy chain of corresponding BCMA antibody, which carries a “Hole” mutation in Fc region to generate a heterodimeric antibody and carries L234A and L235A mutations in Fc CH3; 2) the heavy chain of corresponding CD3 antibody, which carries a “knob” mutation in Fc region to generate a heterodimeric antibody and carries L234A and L235A mutations in Fc CH3; 3) the light chain of corresponding CD3 antibody, respectively. The “knob” mutation in the Fc region of human IgGI consists of T366W, and the “Hole” mutation consists of T366S, L368A and Y407V. In addition, the antibody comprises S354C in the Fc region of “knob” and Y349C of “Hole” to form a disulfide bond to increase stability and heterodimeric antibody yield. Tables 5-7 below shows the sequence information of the bispecific antibodies of the present disclosure and positive control 2, respectively.

TABLE 5
Sequence information of BCMA chain of PR003178 bispecific
antibody

| | |
|---|---|
| Sequence of | EVQLVETGGGLIQPGGSLRLSCAASGFTVSDNYMTWVRQAPGKG |
| PR003178 | LEWVSVIFSGGNTYYADSVKGRFTISRDNAKNTLYLQMNSLRAE |
| HC2 | DTALYYCARRNYDDTRGTDVFDIWGQGTMVTVSSGGGGSGGGG |
| | SGGGGSEVQLVETGGGLIQPGGSLRLSCAASGFTVSDNYMTWVR |
| | QAPGKGLEWVSVIFSGGNTYYADSVKGRFTISRDNAKNTLYLQM |
| | NSLRAEDTALYYCARRNYDDTRGTDVFDIWGQGTMVTVSSASEP |
| | KSSDKTHTCPPCPAPEAAGGPSVFLFPPKPKDTLMISRTPEVTCVV |
| | VDVSHEDPEVKFNWYVDGVEVHNAKTKPREEQYNSTYRVVSVL |
| | TVLHQDWLNGKEYKCKVSNKALPAPIEKTISKAKGQPREPQVCT |
| | LPPSREEMTKNQVSLSCAVKGFYPSDIAVEWESNGQPENNYKTTP |
| | PVLDSDGSFFLVSKLTVDKSRWQQGNVFSCSVMHEALHNHYTQK |
| | SLSLSPGK (SEQ ID NO: 8) |
| Sequence of | EVQLVETGGGLIQPGGSLRLSCAASGFTVSDNYMTWVRQAPGKG |
| PR003178 | LEWVSVIFSGGNTYYADSVKGRFTISRDNAKNTLYLQMNSLRAE |
| VH2 | DTALYYCARRNYDDTRGTDVFDIWGQGTMVTVSS (SEQ ID NO: |
| | 59) |
| PR003178 | GFTVSDNYMT (SEQ ID NO: 22) |
| VH2 CDR1 | |
| (Combined) | |
| PR003178 | VIFSGGNTYYADSVKG (SEQ ID NO: 33) |
| VH2 CDR2 | |
| (Combined) | |
| PR003178 | RNYDDTRGTDVFDI (SEQ ID NO: 42) |
| VH2 CDR3 | |
| (Combined) | |

TABLE 6
Amino acid sequence information of BCMA chain of PR002299
bispecific antibody

| | |
|---|---|
| PR002299 HC2 | EVQLLESGGGLVQPGGSLRLSCAASGFTFSSYAMIWVRQAPGK |
| | GLEWVSGISESGGSTYYADSVKGRFTISRDNSKNTLYLQMNSLR |
| | AEDTAVYFCVKDLDDILTGYYKDYWGQGTLVTVSSRTGGGGS |
| | KLEVQLLESGGGLVQPGGSLRLSCAASGFTFSSYAMIWVRQAPG |
| | KGLEWVSGISESGGSTYYADSVKGRFTISRDNSKNTLYLQMNSL |
| | RAEDTAVYFCVKDLDDILTGYYKDYWGQGTLVTVSSASEPKSS |
| | DKTHTCPPCPAPEAAGGPSVFLFPPKPKDTLMISRTPEVTCVVVD |
| | VSHEDPEVKFNWYVDGVEVHNAKTKPREEQYNSTYRVVSVLT |
| | VLHQDWLNGKEYKCKVSNKALGAPIEKTISKAKGQPREPQVCT |
| | LPPSREEMTKNQVSLSCAVKGFYPSDIAVEWESNGQPENNYKTT |
| | PPVLDSDGSFFLVSKLTVDKSRWQQGNVFSCSVMHEALHNHYT |
| | QKSLSLSPGK (SEQ ID NO: 7) |
| PR002299 VH2 | EVQLLESGGGLVQPGGSLRLSCAASGFTFSSYAMIWVRQAPGK |
| | GLEWVSGISESGGSTYYADSVKGRFTISRDNSKNTLYLQMNSLR |
| | AEDTAVYFCVKDLDDILTGYYKDYWGQGTLVTVSS (SEQ ID |
| | NO: 60) |
| PR002299 VH2 | GFTFSSYAMI (SEQ ID NO: 26) |
| CDR1 | |
| (Combined) | |
| PR002299 VH2 | GISESGGSTYYADSVKG (SEQ ID NO: 37) |
| CDR2 | |
| (Combined) | |
| PR002299 VH2 | DLDDILTGYYKDY (SEQ ID NO: 41) |
| CDR3 | |
| (Combined) | |

TABLE 7
Amino acid sequence information of positive control 2 (PR002199)

| PR002199 | Amino acid sequence and sequence number |
|---|---|
| PR002199 HC1 | EVQLVESGGGLVQPGRSLRLSCAASGFTFDDYAMHWVRQ |
| | APGKGLEWVSGISWNSGSIGYADSVKGRFTISRDNAKNSLY |
| | LQMNSLRAEDTALYYCAKDSRGYGDYRLGGAYWGQGTL |
| | VTVSSASTKGPSVFPLAPSSKSTSGGTAALGCLVKDYFPEPV |
| | TVSWNSGALTSGVHTFPAVLQSSGLYSLSSVVTVPSSSLGT |
| | QTYICNVNHKPSNTKVDKKVEPKSCDKTHTCPPCPAPEAAG |
| | GPSVFLFPPKPKDTLMISRTPEVTCVVVDVSHEDPEVKFNW |
| | YVDGVEVHNAKTKPREEQYNSTYRVVSVLTVLHQDWLNG |
| | KEYKCKVSNKALGAPIEKTISKAKGQPREPQVYTLPPCREE |
| | MTKNQVSLWCLVKGFYPSDIAVEWESNGQPENNYKTTPPV |
| | LDSDGSFFLYSKLTVDKSRWQQGNVFSCSVMHEALHNHYT |
| | QKSLSLSPGK (SEQ ID NO: 3) |
| PR002199 VH1 | EVQLVESGGGLVQPGRSLRLSCAASGFTFDDYAMHWVRQ |
| | APGKGLEWVSGISWNSGSIGYADSVKGRFTISRDNAKNSLY |
| | LQMNSLRAEDTALYYCAKDSRGYGDYRLGGAYWGQGTL |
| | VTVSS (SEQ ID NO: 61) |
| PR002199 VH1 | GFTFDDYAMH (SEQ ID NO: 23) |
| CDR1 (Combined) | |
| PR002199 VH1 | GISWNSGSIGYADSVKG (SEQ ID NO: 35) |
| CDR2 (Combined) | |
| PR002199 VH1 | DSRGYGDYRLGGAY (SEQ ID NO: 43) |
| CDR3 (Combined) | |
| PR002199 HC2 | EVQLLESGGGLVQPGGSLRLSCAASGFTVSSYGMSWVRQA |
| | PGKGPEWVSGIRGSDGSTYYADSVKGRFTISRDNSKNTLYL |
| | QMNSLRAEDTAVYYCAKQGENDGPFDHRGQGTLVTVSSG |
| | GGGSEVQLLESGGGLVQPGGSLRLSCAASGFTVSSYGMSW |
| | VRQAPGKGPEWVSGIRGSDGSTYYADSVKGRFTISRDNSKN |
| | TLYLQMNSLRAEDTAVYYCAKQGENDGPFDHRGQGTLVT |
| | VSSASEPKSSDKTHTCPPCPAPEAAGGPSVFLFPPKPKDTLM |
| | ISRTPEVTCVVVDVSHEDPEVKFNWYVDGVEVHNAKTKPR |
| | EEQYNSTYRVVSVLTVLHQDWLNGKEYKCKVSNKALGAPI |
| | EKTISKAKGQPREPQVCTLPPSREEMTKNQVSLSCAVKGFY |
| | PSDIAVEWESNGQPENNYKTTPPVLDSDGSFFLVSKLTVDK |
| | SRWQQGNVFSCSVMHEALHNHYTQKSLSLSPGK (SEQ ID |
| | NO: 6) |
| PR002199 VH2 | EVQLLESGGGLVQPGGSLRLSCAASGFTVSSYGMSWVRQA |
| | PGKGPEWVSGIRGSDGSTYYADSVKGRFTISRDNSKNTLYL |
| | QMNSLRAEDTAVYYCAKQGENDGPFDHRGQGTLVTVSS |
| | (SEQ ID NO: 57) |
| PR002199 VH2 | GFTVSSYGMS (SEQ ID NO: 24) |
| CDR1 (Combined) | |
| PR002199 VH2 | GIRGSDGSTYYADSVKG (SEQ ID NO: 36) |
| CDR2 (Combined) | |
| PR002199 VH2 | QGENDGPFDH ( SEQ ID NO: 39) |
| CDR3 (Combined) | |
| PR002199 LC | EIVMTQSPATLSVSPGERATLSCRASQSVSSNLAWYQQKPG |
| | QAPRLLIYGASTRATGIPARFSGSGSGTEFTLTISSLQSEDFA |
| | VYYCQQYNNWPWTFGQGTKVEIKRTVAAPSVFIFPPSDEQL |
| | KSGTASVVCLLNNFYPREAKVQWKVDNALQSGNSQESVTE |
| | QDSKDSTYSLSSTLTLSKADYEKHKVYACEVTHQGLSSPVT |
| | KSFNRGEC (SEQ ID NO: 46) |
| PR002199 VL | EIVMTQSPATLSVSPGERATLSCRASQSVSSNLAWYQQKPG |
| | QAPRLLIYGASTRATGIPARFSGSGSGTEFTLTISSLQSEDFA |
| | VYYCQQYNNWPWTFGQGTKVEIK (SEQ ID NO: 65) |
| PR002199 VL CDR1 | RASQSVSSNLA (SEQ ID NO: 49) |
| (Combined) | |
| PR002199 VL CDR2 | GASTRAT (SEQ ID NO: 52) |
| (Combined) | |
| PR002199 VL CDR3 | QQYNNWPWT (SEQ ID NO: 55) |
| (Combined) | |

### EXAMPLE 6. IN VITRO BINDING CAPACITY ASSAY OF BCMA×CD3 BISPECIFIC ANTIBODY ON NCI-H929 CELL LINE WITH HIGHLY EXPRESSED BCMA WITH FACS

To study the binding activity of the BCMA arm of the BCMA×CD3 bispecific antibody to human BCMA, NCI-H929 cell line with highly expressed BCMA was used for binding experiments with human BCMA at cellular level. Briefly, NCI-H929 cell suspensions were collected. The cell density was adjusted to 1×10⁶ cells/mL, and the cells were inoculated in 96 well V-bottom plates at 100 μL cells/well (Corning, Cat #: 3894), followed by the addition of test antibodies at 100 μL/well, which is gradient diluted at 3 folds of the concentration with 2 folds of the final concentration. Cells were incubated at 4° C. for 2 hours in the dark. Afterwards, cells were washed twice with 100 μL/well of pre-cooled PBS, centrifuged at 500 g for 5 min at 4° C., and followed by discarding the supernatant. 100 μL/well of fluorescent secondary antibody (Alexa Fluor® 488-conjugated AffiniPure Goat Anti-Human IgG, Fcγ Fragment Specific, Jackson, Cat #: 109-545-06, 1:500 dilution) was added and incubated at 4° C. for 1 hour in the dark. Cells were washed twice with 100 μL/well of pre-cooled PBS, centrifuged at 500 g, 4° C. for 5 min, and followed by discarding the supernatant. Finally, the cells were resuspended with 200 μL/well of pre-cooled PBS, and the fluorescence luminescence signal values were read by BD FACS CANTOII.

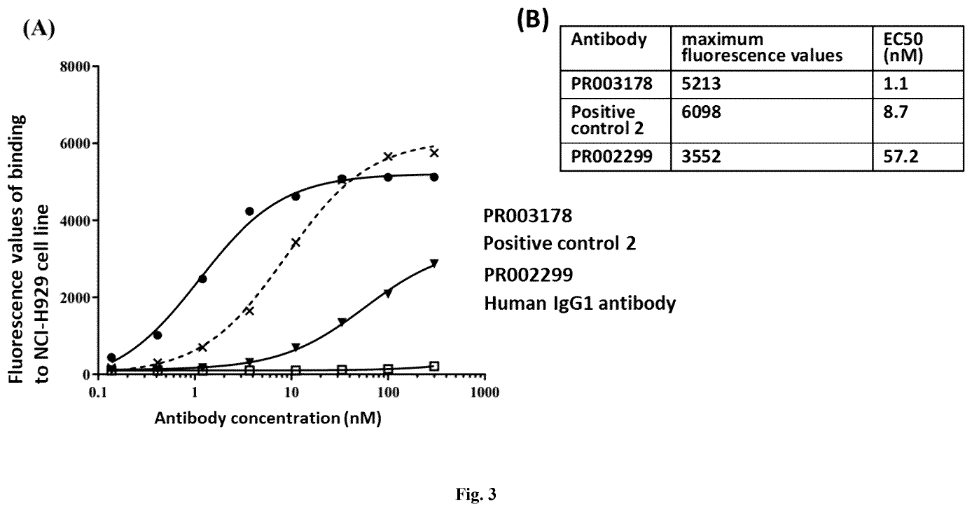

As shown in (A) and (B) of FIG. 3, the binding of BCMA×CD3 bispecific antibody PR003178 to human BCMA molecules on NCI-H929 cell line was superior to that of positive control 2. Specifically, the EC50 of the binding curve of antibody PR003178 was approximately 8 times lower than that of positive control 2. Meanwhile, the binding of antibody PR002299 to human BCMA molecules on NCI-H929 cell line was weaker than that of antibody PR003178 in terms of both the maximum binding fluorescence value and the EC50 of the binding curve.

### EXAMPLE 7. IN VITRO BINDING CAPACITY ASSAY OF BCMA×CD3 BISPECIFIC ANTIBODY ON HEK293T CELL STRAIN OVEREXPRESSING HUMAN AND CYNOMOLGUS MONKEY BCMA WITH FACS

To study the in vitro binding activity of the BCMA arm of the BCMA×CD3 bispecific antibody, HEK293T cell strain overexpressing human BCMA (HEK293T/huBCMA) and HEK293T cell strain overexpressing cynomolgus monkey BCMA (HEK293T/cynoBCMA) were used for antibody binding experiments at cellular level. Briefly, HEK293T/huBCMA cells and HEK293T/cynoBCMA cells were digested and resuspended with DMEM complete medium. The cell density of the two was adjusted to 1×10⁶ cells/mL, respectively. Cells were inoculated at 100 μL cells/well in a 96 well V-bottom plate (Corning, Cat #: 3894), followed by the addition of test antibodies at 100 μL/well, which is gradient diluted at 3 folds of the concentration with 2 folds of the final concentration. Cells were incubated at 4° C. for 1 hour in the dark. Afterwards, the cells were washed twice with 100 μL/well of pre-cooled PBS, centrifuged at 500 g for 5 min at 4° C., and followed by discarding the supernatant. 100 μL/well of fluorescent secondary antibody (Alexa Fluor®) 488-conjugated AffiniPure Goat Anti-Human IgG, Fcγ Fragment Specific, Jackson, Cat #: 109-545-06, 1:500 dilution) was added and incubated for 30 min at 4° C. in the dark. Cells were washed twice with 100 μL/well of pre-cooled PBS, centrifuged at 500 g, 4° C. for 5 min, and followed by discarding the supernatant. Finally, the cells were resuspended with 200 μL/well of pre-cooled PBS, and the fluorescence luminescence signal values were read by BD FACS CANTOII.

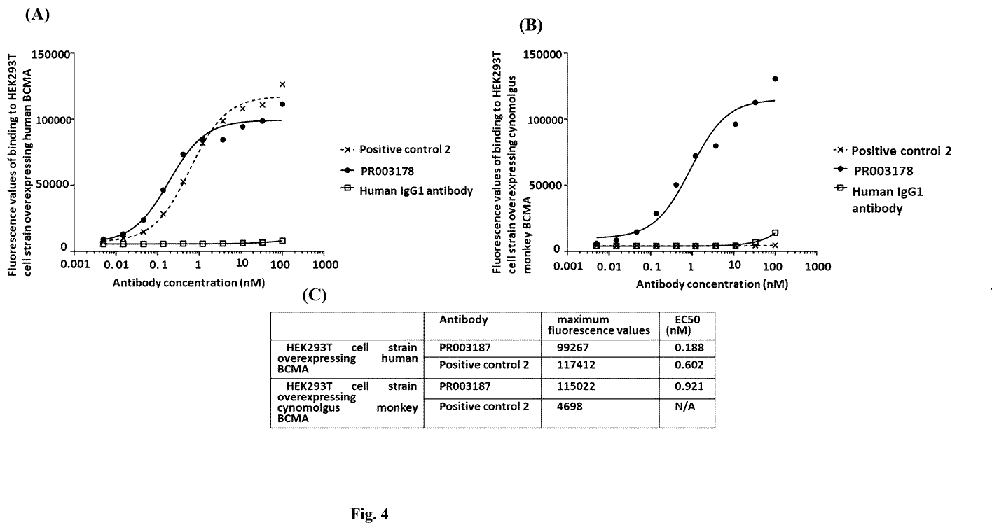

As shown in (A) of FIG. 4, the binding of BCMA×CD3 bispecific antibody PR003178 to human BCMA on HEK293T/huBCMA cells was stronger than that of positive control 2. Specifically, the EC50 of the binding curve of antibody PR003178 was about 5 times lower than that of positive control 2, as shown in (C) of FIG. 4. Meanwhile, as shown in (B) of FIG. 4, PR003178 was able to cross-bind to cynomolgus monkey BCMA on HEK293T/cynoBCMA cells, while positive control 2 has no ability of cross-binding cynomolgus monkey BCMA.

### EXAMPLE 8. IN VITRO BINDING CAPACITY ASSAY OF BCMA×CD3 BISPECIFIC ANTIBODY ON HUMAN AND CYNOMOLGUS MONKEY T CELLS WITH FACS

To study the in vitro binding activity of the CD3 arm of the BCMA×CD3 bispecific antibody, human and cynomolgus monkey T cells were used for antibody binding experiments at cellular level. Briefly, the density of human and cynomolgus monkey T cells was adjusted to 1×10⁶ cells/mL, respectively. Cells were inoculated at 100 μL cells/well in a 96 well V-bottom plate (Corning, Cat #: 3894), followed by the addition of test antibodies at 100 μL/well, which is gradient diluted at 3 folds of the concentration with 2 folds of the final concentration. Cells were incubated at 4° C. for 1 hour in the dark. Afterwards, cells were washed twice with 100 μL/well of pre-cooled PBS, centrifuged at 500 g in 4° C. for 5 min, and followed by discarding the supernatant. 100 μL/fluorescent secondary antibody (Alexa Fluor®) 488-conjugated AffiniPure Goat Anti-Human IgG, Fcγ Fragment Specific, Jackson, Cat #: 109-545-06, 1:500 dilution) was added and incubated for 30 min at 4° C. in the dark. Cells were washed twice with 100 μL/well of pre-cooled PBS, centrifuged at 500 g, 4° C. for 5 min, and followed by discarding the supernatant. Finally, the cells were resuspended with 200 μL/well of pre-cooled PBS, and the fluorescence luminescence signal values were read by BD FACS CANTOII.

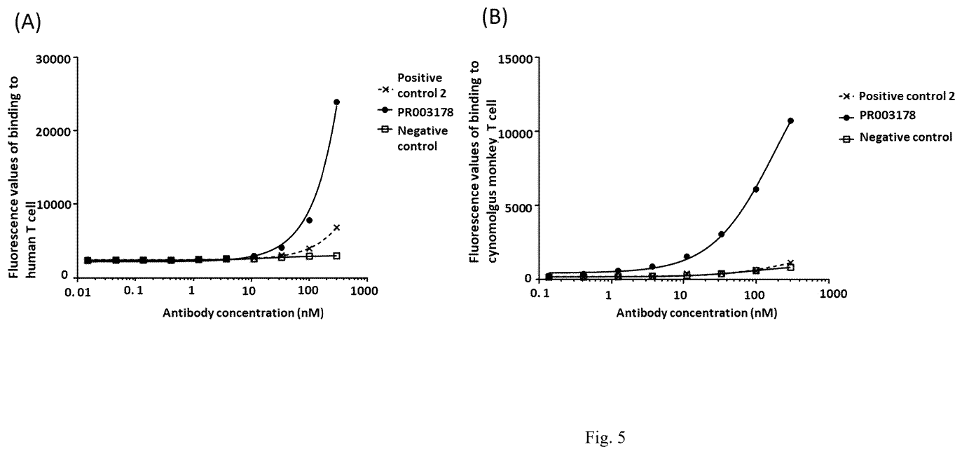

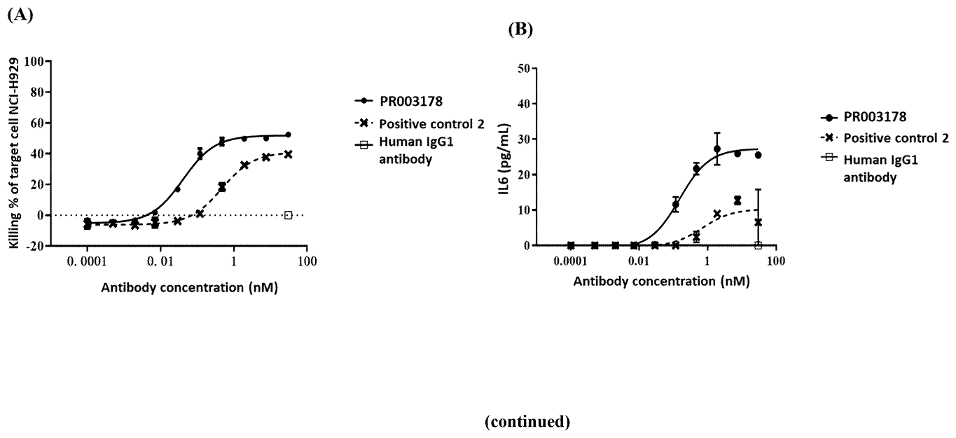

As shown in (A) of FIG. 5, the binding of BCMA×CD3 bispecific antibody PR003178 to human CD3 on human T cells was stronger than that of positive control 2. Specifically, the fluorescence value of PR003178 binding was higher than that of positive control 2 binding at the same antibody concentration. Meanwhile, as shown in (B) of FIG. 5, antibody PR003178 was able to cross-bind to cynomolgus monkey CD3 on cynomolgus monkey T cells, while positive control 2 have no ability of cross-binding cynomolgus monkey BCMA.

### EXAMPLE 9. IN VITRO KILLING EXPERIMENT AND CYTOKINE RELEASE OF BCMA×CD3 BISPECIFIC ANTIBODY AGAINST CELL LINE NCI-H929 HIGHLY EXPRESSING BCMA

To study the in vitro target cell killing ability mediated by BCMA×CD3 bispecific antibody, human PBMC was used as effector cell and NCI-H929 cell line highly expressing BCMA was used as target cell for in vitro killing experiments and cytokine release detection. Specifically, the density of PBMC was adjusted to 1.1×10⁶ cells/mL and the density of NCI-H929 was adjusted to 0.11×10⁶ cells/mL by using RPMI1640/5% FBS medium. Each of the two cell suspensions were inocubated at 90 μL cells/well in a 96 well U-bottom plate (Corning, Cat #: 3799), followed by the addition of test antibodies at 20 μL/well, which is gradient diluted at 4 folds of the concentration with 10 folds of the final concentration, wherein the maximum final concentration of antibody is 30 nM, and each antibody has 10 concentrations in total with the final effector-target ratio as 10:1, and two replicates were set. Meanwhile, different controls were set in the plate: ER (0): NCI-H929+PBMC+RPMI1640/5% FBS medium; ESR: PBMC+RPMI1640/5% FBS medium; TSR: NCI-H929+RPMI1640/5% FBS medium; CMB: RPMI1640/5% FBS medium only; TMR: NCI-H929 +RPMI1640/5% FBS medium+lysis buffer (added after incubation for 24 hours); VCC: RPMI1640/5% FBS medium+lysis buffer (added after incubation for 24 hours). The 96 well plates were incubated for 24 hours in a CO₂ incubator at 37° C. After incubation, 50 μl of supernatant was taken into the 96 well plate (Corning, Cat #: 3599), and 50 μl of cytotoxicity test reagent (CytoTox 96® Non-Radioactive Cytotoxicity Test Kit, Promega, USA, cat #G1780) was added and incubated for 30 min at room temperature. The termination solution was added to terminate the reaction, and the OD490 values were read with Enspire (Perkin Elmer). The OD490 values were used to calculate the killing effect according to the following formula, % of specific killing={(ER−CMB)−[(ESR−CMB)+(TSR−CMB)]}/(TMR−VCC) 100%. Cell culture supernatants were collected to detect the release of cytokines IL-6 and TNF-α. ELISA detection method refers to IL-6 (IL-6 Human Uncoated ELISA Kit, Thermo, Cat #: 88-7066-88) and TNF-α (TNF alpha Human Uncoated ELISA Kit, Thermo, Cat #: 88-7346-88) kit operating instructions.

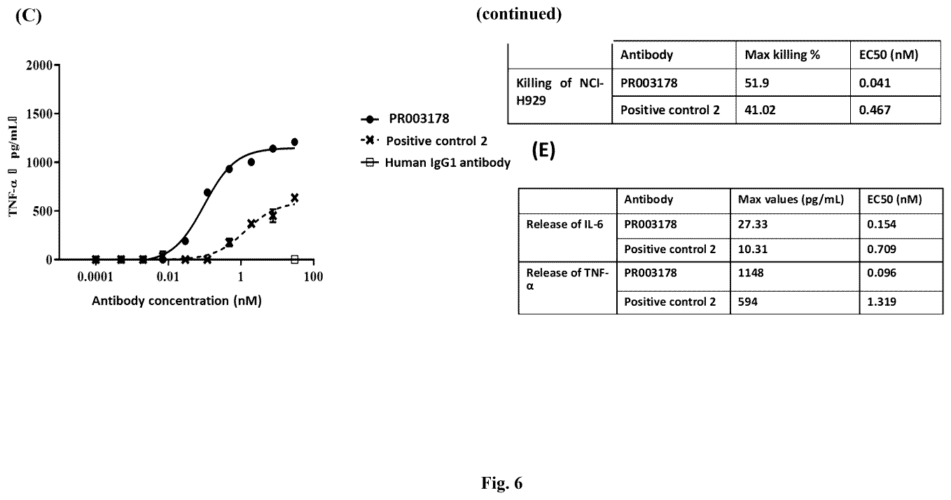

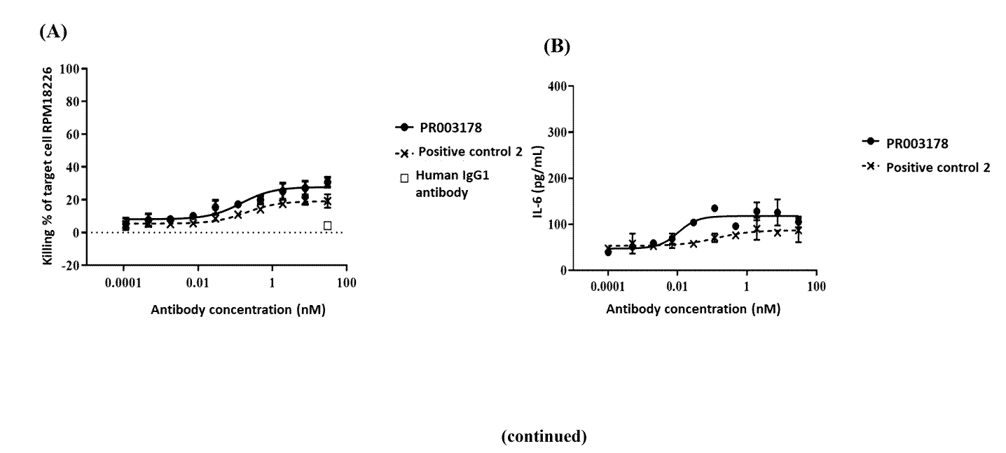

As shown in FIG. 6, the killing effect of PBMC mediated by BCMA×CD3 bispecific antibody PR003178 against cell line NCI-H929 with highly expressed BCMA was superior to that of positive control 2 (A). Specifically, the killing percentage of PR003178 at high concentration was higher than that of positive control 2, and the EC50 of the killing curve of PR003178 was 10 times less than that of positive control 2 (D). Meanwhile, the release of cytokines IL-6 and TNF-α produced in the killing system of PR003178 was higher than that of positive control 2 (B, C, E).

### EXAMPLE 10. IN VITRO KILLING EXPERIMENT AND CYTOKINE RELEASE OF BCMA×CD3 BISPECIFIC ANTIBODY AGAINST CELL LINE RPMI8226 LOW EXPRESSING BCMA

To study the in vitro target cell killing ability mediated by BCMA×CD3 bispecific antibody, human PBMC were used as effector cell and the cell line RPMI8226 (CAS, CAS Cell Bank) which low expresses BCMA was used as target cell for in vitro killing experiments and cytokine release detection. Specifically, the density of PBMC was adjusted to 1.1×10⁶ cells/mL and the density of RPMI8226 was adjusted to 0.11×10⁶ cells/mL by using RPMI1640/5% FBS medium. Each of the two cell suspensions were inoculated at 90 μL cells/well in a 96 well U-bottom plate (Corning, Cat #: 3799), followed by the addition of test antibodies at 20 μL/well, which is gradient diluted at 4 folds of the concentration with 10 folds of the final concentration, wherein the maximum final concentration of antibody was 30 nM, and each antibody has 10 concentrations in total with the final effector-target ratio as 10:1, and two replicates were set. Meanwhile, different controls were set in the plate: ER (0): RPMI8226+PBMC+RPMI1640/5% FBS medium; ESR: PBMC+RPMI1640/5% FBS medium; TSR: RPMI8226+RPMI1640/5% FBS medium; CMB: RPMI1640/5% FBS medium only; TMR: RPMI8226+RPMI1640/5% FBS medium+lysis buffer (added after incubation for 24 hours); VCC: RPMI1640/5% FBS medium+lysis buffer (added after incubation for 24 hours). The 96 well plates were incubated at 37° C. in a CO₂ incubator for 24 hours. After incubation, 50 μl of supernatant was added to the 96 well plate (Corning, Cat #: 3599), and 50 μl of cytotoxicity test reagent (CytoTox 96® Non-Radioactive Cytotoxicity Test Kit, Promega, USA, Cat #: G1780) was added and incubated for 30 min at room temperature. The termination solution was added to terminate the reaction, and the OD490 values were read with Enspire (Perkin Elmer). The OD490 values were used to calculate the killing effect according to the following formula, % of specific killing={(ER−CMB)−[(ESR−CMB)+(TSR−CMB)]}/(TMR−VCC) 100%. Cell culture supernatants were collected to detect the release of cytokines IL-6 and TNF-α. ELISA detection method refers to IL-6 (IL-6 Human Uncoated ELISA Kit, Thermo, Cat #: 88-7066-88) and TNF-α (TNF alpha Human Uncoated ELISA Kit, Thermo, Cat #: 88-7346-88) kit operating instructions.

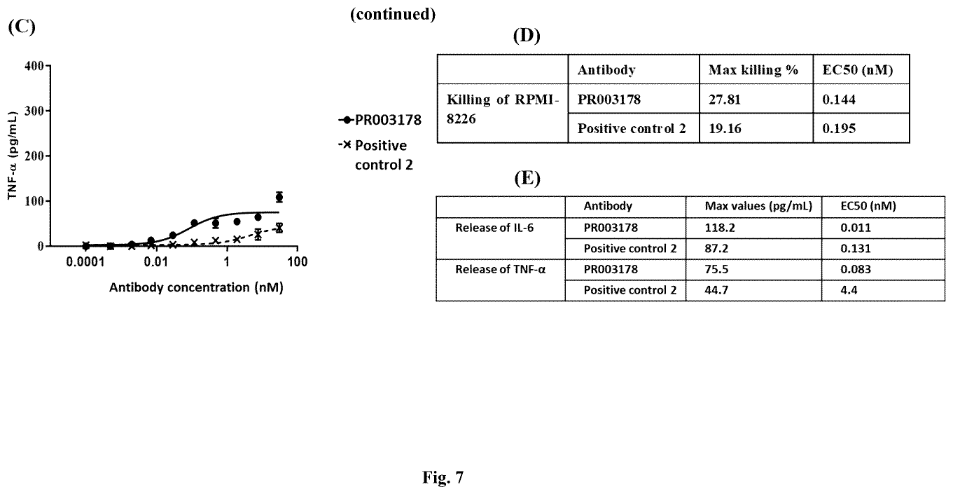

As shown in FIG. 7, the killing effect of PBMC mediated by BCMA×CD3 bispecific antibody PR003178 against cell line with low BCMA expression was better than that of positive control 2 (A). Specifically, the target cell killing percentage of PR003178 at high concentration was higher than that of positive control 2 (D). Meanwhile, the release of cytokines IL-6 and TNF-a produced in the killing system of PR003178 was higher than that of positive control 2 (B, C, E).

EXAMPLE 11. BINDING OF BCMA×CD3 BISPECIFIC ANTIBODY TO CELL LINE HL-60 WITHOUT BCMA EXPRESSION

To study the in vitro binding specificity of BCMA×CD3 bispecific antibody, cell line HL-60 (ATCC® CCL-240™) without human BCMA expression was used for antibody binding experiments at cellular level. Briefly, HL-60 cell suspensions were collected. The cell density was adjusted to 1×10⁶ cells/mL, respectively, and the cells were inoculated at 100 μL cells/well in 96 well V-bottom plates (Corning, Cat #: 3894), followed by the addition of test antibodies at 100 μL/well, which is gradient diluted at 3 folds of the concentration with 2 folds of the final concentration. The maximum final concentration of antibody is 300 nM, and each antibody has 4 concentrations. Cells were incubated at 4° C. for 1 hours in the dark. Afterwards, the cells were washed twice with 100 μL/well of pre-cooled PBS, centrifuged at 500 g in 4° C. for 5 min, and followed by discarding the supernatant. 100 μL/well of fluorescent secondary antibody (Alexa Fluor® 488-conjugated AffiniPure Goat Anti-Human IgG, Fcγ Fragment Specific, Jackson, Cat #: 109-545-06, 1:500 dilution) was added and incubated for 30 min at 4° C. in the dark. Cells were washed twice with 100 μL/well of pre-cooled PBS, centrifuged at 500 g for 5 min at 4° C., and followed by discarding the supernatant. Finally, the cells were resuspended with 200 μL/well of pre-cooled PBS, and the fluorescence luminescence signal values were read by BD FACS CANTOII.

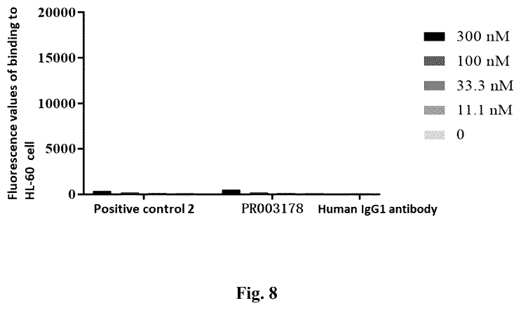

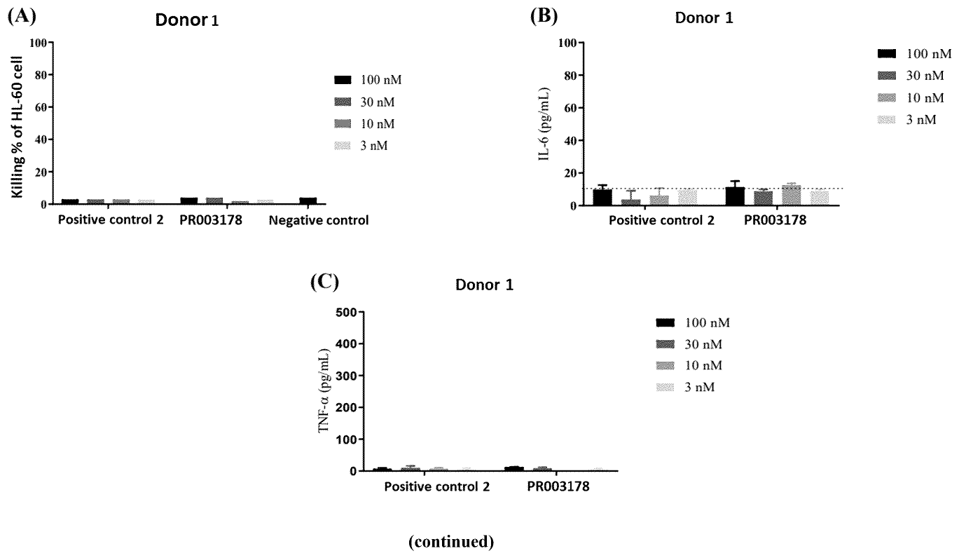

As shown in FIG. 8, BCMA×CD3 bispecific antibody PR003178 and positive control 2 failed to bind non-specifically to the BCMA-negative cell line HL-60 at 300 nM, 100 nM, 33.3 nM and 11.1 nM.

### EXAMPLE 12. DETECTION OF NON-SPECIFIC KILLING AND CYTOKINE RELEASE OF BCMA×CD3 BISPECIFIC ANTIBODY AGAINST BCMA-NEGATIVE CELL LINE HL60 WITH FACS

To study the specificity of the target cell killing ability mediated by BCMA×CD3 bispecific antibody in vitro, human PBMC was used as effector cell and cell line HL-60 without expressing BCMA was used as target cell for in vitro killing experiments and cytokine release detection. Specifically, target cell HL-60 was labeled with 0.5 μM of CFSE dye. The density of PBMC from three donors was adjusted to 1.1×10⁶ cells/mL, and the density of CFSE-labeled HL-60 cells was adjusted to 0.11×10⁶ cells/mL by using RPMI1640/5% FBS medium. Each of the two cell suspensions were inoculated at 90 μL cells/well in a 96 well U-bottom plate (Corning, Cat #: 3799), followed by the addition of test antibodies at 20 μL/well, which has a concentration 10 fold of the final concentration, wherein the final concentrations of antibodies were 100 nM, 30 nM, 10 nM and 3 nM, four concentrations in total. The final effector-target ratio was 10:1. Set two replicates. The 96 well plates were incubated in a CO₂ incubator at 37° C. for 24 hours. After incubation, cell culture supernatants were collected to detect the release of cytokines IL-6 and TNF-α. ELISA detection method refers to IL-6 (IL-6 Human Uncoated ELISA Kit, Thermo, Cat #: 88-7066-88) and TNF-α (TNF alpha Human Uncoated ELISA Kit, Thermo, Cat #: 88-7346-88) kit operating instructions. The cultured cells were transferred to 96 well V-bottom plates (Corning, Cat #: 3894), stained with 100 μL/well of 7-AAD (BD, Cat #: 559925, 1:100) staining system, and incubated for 30 min at room temperature. The reaction was terminated by adding 100 μL/well of PBS. Detection was performed using BD FACS CANTOII. The killing effect was calculated according to the following formula, % of specific killing=7-AAD+CFSE+percentage/(7-AAD+CFSE+percentage+7-AAD-CFSE+percentage)*100%.

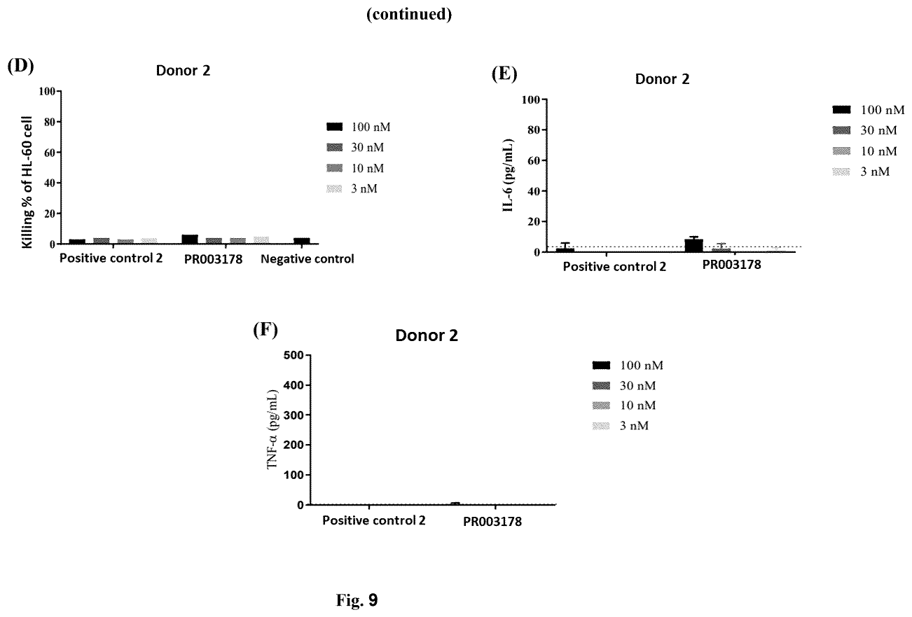

As shown in A-E of FIG. 9, the BCMA×CD3 bispecific antibody PR003178 and positive control 2 could not mediate the non-specific killing of PBMC from two donors to BCMA negative cell line HL-60 at 100 nM, 30 nM, 10 nM and 3 nM (A, D of FIG. 9), and non-specific cytokines IL-6 (B, E of FIG. 9) and TNF-α (C, F of FIG. 9) could not be detected in this system.

EXAMPLE 13: EVALUATION OF ANTITUMOR EFFICACY OF BCMA×CD3 BISPECIFIC ANTIBODIES IN NCI-H929/HUMAN PBMC MICE MODEL

To evaluate the in vivo antitumor efficacy of BCMA×CD3 bispecific antibodies, 6-8 week old female NCG mice were used. All mice were injected intraperitoneally (i.p.) with 3×10⁶ human PBMC three days before tumor cell inoculation, and then each experimental mouse was inoculated subcutaneously with 5×10⁶ NCI-H929 cells on the day of tumor cell inoculation. The cells were resuspended in a mixture of PBS and Matrigel (1:1) (0.1 mL per mouse). Mice were randomly grouped into 6 mice in each group when the tumor volume reached 90 mm³. After grouping, the drug diluted with PBS at a specific concentration was administered intravenously (i.v.) once a week in a total of 2 (QW*2), and PBS was used as a mock group. Tumor volume and mice body weight were measured on day 3, 7, 10 and 14 days after the initial administration. Tumor size was calculated by the formula: tumor volume (mm³)=0.5×(tumor length diameter×tumor short diameter²).

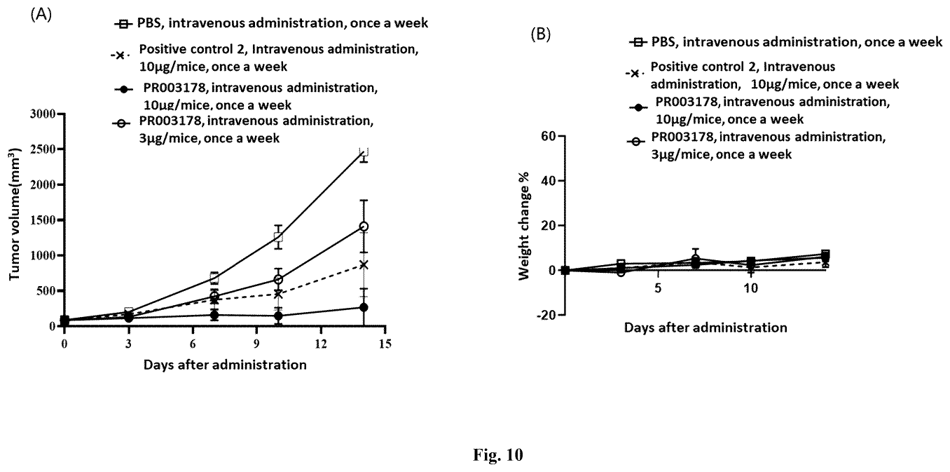

As shown in (A) of FIG. 10, BCMA×CD3 bispecific antibody PR003178 was effective in inhibiting tumor growth at both 10 μg/mouse and 3 μg/mouse, and the efficacy of the 10 μg/mouse group is superior to that of the 3 μg/mouse group. Also, the efficacy of PR003178 at the 10 μg/mouse dose is superior to that of positive control 2 at the same dose. As shown in (B) of FIG. 10, the body weight changes of the mice in each group are within the normal range.

### EXAMPLE 14. DETECTION FOR AFFINITY OF PR003178 TO HUMAN CD3E/G RECOMBINANT PROTEIN, CYNOMOLGUS MONKEY CD3E/G RECOMBINANT PROTEIN, HUMAN BCMA RECOMBINANT PROTEIN AND CYNOMOLGUS MONKEY BCMA RECOMBINANT PROTEIN WITH BIACORE

HBS-EP+(10 mM HEPES, 150 mM NaCl, 3 mM EDTA and 0.05% P20, pH 7.4, GE Healthcare, Cat #BR-1006-69) was used as running buffer for the entire test, and Series S CM5 (GE Healthcare, Cat #BR-1005-30) was used as the experimental chip. Human BCMA-Fc, Avitag recombinant protein (ACRO, Cat. BC7-H82F0), cynomolgus monkey BCMA-Fc, Avitag (ACRO, Cat. BCA-C82F4) were purchased from ACRO, human CD3e/g ECD-hFc recombinant protein (Lot.20190508002), cynomolgus monkey CD3e/g ECD-hFc recombinant protein and human (Lot.2018040001) were purchased from ChemPartner.

### 14.1 Coupling of Antigens

The flow rate was set as 10 μl/min and four antigens were coupled on four channels on two CM5: 1) the injection time was set as 300 s, and 50 mM of NHS and 200 mM of EDC were freshly mixed at 1:1 volume ratio and injected into four channels; 2) human BCMA-Fc, Avitag recombinant protein, cynomolgus monkey BCMA-FC, Avitag, human CD3e/g ECD-hFc recombinant protein and cynomolgus CD3e/g ECD-hFc recombinant protein were all diluted to lug/ml with sodium acetate (Cat #BR-1003-50) (pH 4.5), and injected into channel 2, 3 and 4 of chip 1 and channel 2 of chip 2, respectively; 3) 1 M of ethanolamine (pH8.5) was injected for 300 s to block the remaining active carboxyl groups on the chips surface. After blockage, the instrument was continued to be equilibrated with 1× HBS− EP+ buffer for two hours. The final coupling amounts of human BCMA-Fc, Avitag recombinant protein, cynomolgus monkey BCMA-Fc, Avitag, human CD3e/g ECD-hFc recombinant protein and cynomolgus monkey CD3e/g ECD-hFc recombinant protein were 120 RU, 70 RU, 200 RU and 290 RU, respectively.

### 14.2 Affinity Determination

A multi-cycle kinetic mode was set, and each cycle included the binding of PR003178 as well as the regeneration of the chip. PR003178 with two-fold gradient dilution was injected into four channels at a flow rate of 30 μl/min. A binding time of 180 s and a dissociation time of 400 s or 600 s were set. Finally, 10 mM of glycine-hydrochloric acid (pH 1.5) (GE Life Sciences, Cat #BR-1003-54) was injected at the same flow rate for 60 s to regenerate the chip.

The experimental results were analyzed by using Biacore T200 analysis software, and channel 1 was deducted as reference channel, and a 1:1 kinetic fit model was selected as the analysis model.

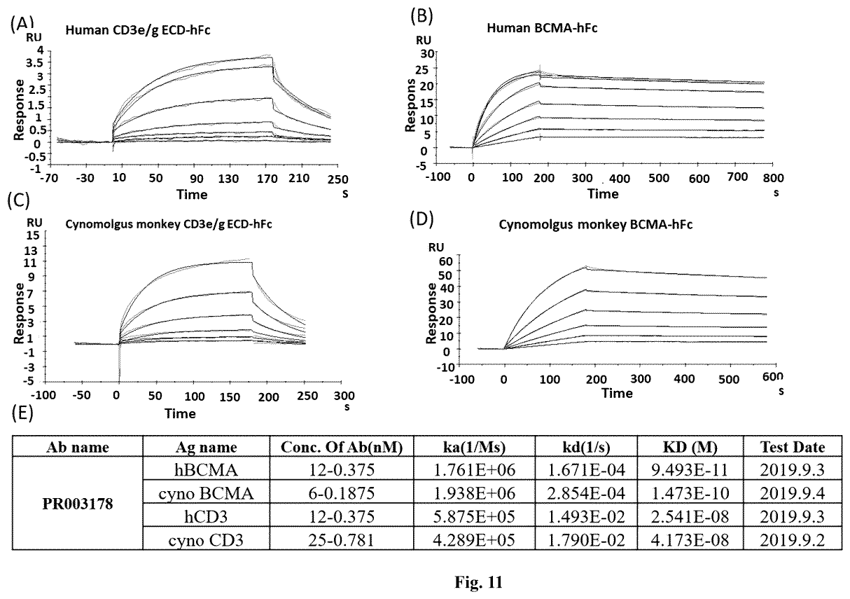

As shown in (B) and (D) of FIG. 11, BCMA×CD3 bispecific antibody PR003178 binds to human BCMA with high affinity, and the affinity to human BCMA-Fc was about 9.493 E-11 M with excellent cross-binding activity to human and cynomolgus monkey BCMA. As shown in (A) and (C) of FIG. 11, BCMA×CD3 bispecific antibody PR003178 binds to human CD3 e/g ECD-hFc with the affinity of about 2.541E-08 Mand the antibody has excellent cross-binding activity to human and cynomolgus monkey CD3 e/g ECD-hFc. The detailed data including Ka (1/Ms), Kd (1/s) and KD of affinity determination for BCMA×CD3 bispecific antibody PR003178 to BCMA and CD3 e/g ECD-hFc is shown in (E) of FIG. 11.

What is claimed is:

1. A bispecific antibody comprising a first protein functional region targeting BCMA and a second protein functional region targeting CD3, wherein the first protein functional region comprises two heavy chain variable regions; wherein at least one of the two heavy chain variable regions of the first protein functional region comprises HCDR1, HCDR2 and HCDR3, wherein the HCDR1, HCDR2 and HCDR3 comprise the amino acid sequences of SEQ ID NO: 22, SEQ ID NO: 33 and SEQ ID NO: 42, respectively; or the amino acid sequences of SEQ ID NO: 26, SEQ ID NO: 37 and SEQ ID NO: 41, respectively.

2. The bispecific antibody of claim 1, wherein at least one of the heavy chain variable regions comprises an amino acid sequence of SEQ ID NO: 59 or an amino acid sequence of SEQ ID NO: 60.

3. The bispecific antibody of claim 1 comprising an Fc fragment, wherein the Fc fragment is an Fc fragment of hIgG1, hIgG2, hIgG3, hIgG4, or a mutant thereof.

4. The bispecific antibody of claim 3, wherein the first protein functional region targeting BCMA comprises an amino acid sequence of SEQ ID NO: 1, SEQ ID NO: 7 or SEQ ID NO: 8.

5. The bispecific antibody of claim 1, wherein the two heavy chain variable regions are linked by (G₄S)ₙ, wherein n is a natural number.

6. The bispecific antibody of claim 5, wherein n is 3 or 4.

7. The bispecific antibody of claim 1, wherein the second protein functional region comprises a heavy chain variable region and a light chain variable region, wherein the heavy chain variable region comprises HCDR1, HCDR2 and HCDR3 with amino acid sequences of SEQ ID NO: 25, SEQ ID NO: 38 and SEQ ID NO: 44, respectively; and wherein the light chain variable region comprises LCDR1, LCDR2 and LCDR3 with amino acid sequences of SEQ ID NO: 50, SEQ ID NO: 53 and SEQ ID NO: 56, respectively.

8. The bispecific antibody of claim 7, wherein the heavy chain variable region of the second protein functional region comprises an amino acid sequence selected from SEQ ID NO: 63, SEQ ID NO: 62, or a mutant thereof, and the light chain variable region of the second protein functional region comprises an amino acid sequence selected from SEQ ID NO: 66 or a mutant thereof.

9. The bispecific antibody of claim 1, comprising a heavy chain constant region and a light chain constant region, wherein the heavy chain constant region comprises a human heavy chain constant region and the light chain constant region comprise a human light chain constant region.

10. The bispecific antibody of claim 9, wherein the human light chain constant region is a human λ or κ light chain constant region, and the human heavy chain constant region is a heavy chain constant region of hIgG1, hIgG2, hIgG3, hIgG4, or a mutant thereof.

11. The bispecific antibody of claim 10, wherein, the mutant of the human heavy chain constant region is selected from (1) L234A/L235A mutation; (2) knob mutation T366W and hole mutations T366S, L368A and Y407V; or (3) knob mutation S354C and hole mutation Y349C.

12. The bispecific antibody of claim 1, wherein the first protein functional region comprises a heavy chain comprising an amino acid sequence of SEQ ID NO: 8 or SEQ ID NO: 7, and the second protein functional region comprises a heavy chain comprising an amino acid sequence of SEQ ID NO: 5 or SEQ ID NO: 4 and a light chain comprising an amino acid sequence of SEQ ID NO: 47.

13. An isolated nucleic acid encoding the bispecific antibody of claim 1.

14. An expression vector comprising the isolated nucleic acid of claim 13.

15. A host cell comprising the expression vector of claim 14.

16. An antibody drug conjugate comprising the bispecific antibody of claim 1, and a cytotoxic agent.

17. A pharmaceutical composition comprising the bispecific antibody of claim 1 and a pharmaceutically acceptable carrier.

18. A pharmaceutical composition comprising the antibody drug conjugate of claim 16, and a pharmaceutically acceptable carrier.

19. A method for treating and/or preventing cancer in a subject in need thereof, comprising: administering an effective amount of the bispecific antibody of claim 1 to the subject.

20. A method for treating and/or preventing cancer in a subject in need thereof, comprising: administering an effective amount of the antibody drug conjugate of claim 16 to the subject.
